SEQUENCE LISTING (64 sequences)

SEQ ID NO: 1 - DNA, 121 bp, Pisum sativum
  misc_feature 1..121: note = Description of sequence: Oligonucleotide, marker at position 363885584 on pea chromosome 2.
  source 1..121: mol_type = genomic DNA
  variation 61: note = site of single nucleotide polymorphism (SNP)
gatatcttttccaatgacatactcatcaagtcagatggcaggacaagacctttcaaagaaatcatgtgaaattcatcaagattcaccagactaacacaaagtaaacaattatacagaaaac

SEQ ID NO: 2 - DNA, 121 bp, Pisum sativum
  misc_feature 1..121: note = Description of sequence: Oligonucleotide, marker at position 363885584 on pea chromosome 2.
  source 1..121: mol_type = genomic DNA
  variation 61: note = site of single nucleotide polymorphism (SNP)
gatatcttttccaatgacatactcatcaagtcagatggcaggacaagacctttcaaagaagtcatgtgaaattcatcaagattcaccagactaacacaaagtaaacaattatacagaaaac

SEQ ID NO: 3 - DNA, 121 bp, Pisum sativum
  misc_feature 1..121: note = Description of sequence: Oligonucleotide, marker at position 113590538 on pea chromosome 3.
  source 1..121: mol_type = genomic DNA
  variation 61: note = site of single nucleotide polymorphism (SNP)
tactatggacgatttcatgagttcgtttttactaaactgtgttgcttttgaacaatgttacagtggttgttctatgaagtatttcacaacctatgtaacattattggattgtttgataaat

SEQ ID NO: 4 - DNA, 121 bp, Pisum sativum
  misc_feature 1..121: note = Description of sequence: Oligonucleotide, marker at position 113590538 on pea chromosome 3.
  source 1..121: mol_type = genomic DNA
  variation 61: note = site of single nucleotide polymorphism (SNP)
tactatggacgatttcatgagttcgtttttactaaactgtgttgcttttgaacaatgttatagtggttgttctatgaagtatttcacaacctatgtaacattattggattgtttgataaat

SEQ ID NO: 5 - DNA, 121 bp, Pisum sativum
  misc_feature 1..121: note = Description of sequence: Oligonucleotide, marker at position 3563293 on pea chromosome 3.
  source 1..121: mol_type = genomic DNA
  variation 61: note = site of single nucleotide polymorphism (SNP)
caaaagtatttgagcagtcatgactaaaacctacagcacaaataaccaattgagtagttgtggtacaaaaattcaaaaacacaggtggccgaagaagtatagctacaaggtcaagaaaatc

SEQ ID NO: 6 - DNA, 121 bp, Pisum sativum
  misc_feature 1..121: note = Description of sequence: Oligonucleotide, marker at position 3563293 on pea chromosome 3.
  source 1..121: mol_type = genomic DNA
  variation 61: note = site of single nucleotide polymorphism (SNP)
caaaagtatttgagcagtcatgactaaaacctacagcacaaataaccaattgagtagttgcggtacaaaaattcaaaaacacaggtggccgaagaagtatagctacaaggtcaagaaaatc

SEQ ID NO: 7 - DNA, 121 bp, Pisum sativum
  misc_feature 1..121: note = Description of sequence: Oligonucleotide, marker at position 427407003 on pea chromosome 2.
  source 1..121: mol_type = genomic DNA
  variation 61: note = site of single nucleotide polymorphism (SNP)
cataccatagatacatgctttaagaaatggaacacaaagatcaccttagaaatgatgtgctagctacaaagatcactttatgatttgaagcagacaggtaggaaatgaaacacaaaacata

SEQ ID NO: 8 - DNA, 121 bp, Pisum sativum
  misc_feature 1..121: note = Description of sequence: Oligonucleotide, marker at position 427407003 on pea chromosome 2.
  source 1..121: mol_type = genomic DNA
  variation 61: note = site of single nucleotide polymorphism (SNP)
cataccatagatacatgctttaagaaatggaacacaaagatcaccttagaaatgatgtgccagctacaaagatcactttatgatttgaagcagacaggtaggaaatgaaacacaaaacata

SEQ ID NO: 9 - DNA, 110 bp, Pisum sativum
  misc_feature 1..110: note = Description of sequence: Oligonucleotide, marker at position 27314266 on pea chromosome 6.
  source 1..110: mol_type = genomic DNA
  variation 50: note = site of single nucleotide polymorphism (SNP)
tgtgatgtatcagtgtgacaaacagcaacagctttaactctatttccatcaacaccttccaatggcaccgagtaaactttagttgtgtctgttttatgacagtagaaaac

SEQ ID NO: 10 - DNA, 110 bp, Pisum sativum
  misc_feature 1..110: note = Description of sequence: Oligonucleotide, marker at position 27314266 on pea chromosome 6.
  source 1..110: mol_type = genomic DNA
  variation 50: note = site of single nucleotide polymorphism (SNP)
tgtgatgtatcagtgtgacaaacagcaacagctttaactctatttccattaacaccttccaatggcaccgagtaaactttagttgtgtctgttttatgacagtagaaaac

SEQ ID NO: 11 - DNA, 121 bp, Pisum sativum
  misc_feature 1..121: note = Description of sequence: Oligonucleotide, marker at position 562131030 on pea chromosome 5.
  source 1..121: mol_type = genomic DNA
  variation 61: note = site of single nucleotide polymorphism (SNP)
cttttctaaaactaagttcaccagatttcccttacttgaaaaattggatctatgtgtttcggatgatttgaaatctattacaatttataaccattgtcttcagagaattgctttaaaagga

SEQ ID NO: 12 - DNA, 121 bp, Pisum sativum
  misc_feature 1..121: note = Description of sequence: Oligonucleotide, marker at position 562131030 on pea chromosome 5.
  source 1..121: mol_type = genomic DNA
  variation 61: note = site of single nucleotide polymorphism (SNP)
cttttctaaaactaagttcaccagatttcccttacttgaaaaattggatctatgtgtttcagatgatttgaaatctattacaatttataaccattgtcttcagagaattgctttaaaagga

SEQ ID NO: 13 - DNA, 121 bp, Pisum sativum
  misc_feature 1..121: note = Description of sequence: Oligonucleotide, marker at position 314710599 on pea chromosome 5.
  source 1..121: mol_type = genomic DNA
  variation 61: note = site of single nucleotide polymorphism (SNP)
acaaacatgccacttggcagctaaccctgtgattgcttgtgctttgtgtgttatctttgtcgcacttcggaacatgatggacaacgcagaaagaagcgtcaccgaacacaactgcaacaac

SEQ ID NO: 14 - DNA, 121 bp, Pisum sativum
  misc_feature 1..121: note = Description of sequence: Oligonucleotide, marker at position 314710599 on pea chromosome 5.
  source 1..121: mol_type = genomic DNA
  variation 61: note = site of single nucleotide polymorphism (SNP)
acaaacatgccacttggcagctaaccctgtgattgcttgtgctttgtgtgttatctttgtagcacttcggaacatgatggacaacgcagaaagaagcgtcaccgaacacaactgcaacaac

SEQ ID NO: 15 - DNA, 121 bp, Pisum sativum
  misc_feature 1..121: note = Description of sequence: Oligonucleotide, marker at position 25529885 on pea chromosome 2.
  source 1..121: mol_type = genomic DNA
  variation 61: note = site of single nucleotide polymorphism (SNP)
ccgaagccattaaagagcaaaattgggtcaatgatcatgaagtgtttgagtttggttggtctccgtctgcatttggaagatgggaagaagaatttggatgaagatgaaggttggggaagag

SEQ ID NO: 16 - DNA, 121 bp, Pisum sativum
  misc_feature 1..121: note = Description of sequence: Oligonucleotide, marker at position 25529885 on pea chromosome 2.
  source 1..121: mol_type = genomic DNA
  variation 61: note = site of single nucleotide polymorphism (SNP)
ccgaagccattaaagagcaaaattgggtcaatgatcatgaagtgtttgagtttggttggtttccgtctgcatttggaagatgggaagaagaatttggatgaagatgaaggttggggaagag

SEQ ID NO: 17 - DNA, 121 bp, Pisum sativum
  misc_feature 1..121: note = Description of sequence: Oligonucleotide, marker at position 230113430 on pea chromosome 5.
  source 1..121: mol_type = genomic DNA
  variation 61: note = site of single nucleotide polymorphism (SNP)
ctttccttaaactctctgtccattcatctggatcaaaactcatcaactgtttcactgtcaactcaaatgttatctgcaacaaacaaatatattcatcatttttactactataccatacctg

SEQ ID NO: 18 - DNA, 121 bp, Pisum sativum
  misc_feature 1..121: note = Description of sequence: Oligonucleotide, marker at position 230113430 on pea chromosome 5.
  source 1..121: mol_type = genomic DNA
  variation 61: note = site of single nucleotide polymorphism (SNP)
ctttccttaaactctctgtccattcatctggatcaaaactcatcaactgtttcactgtcagctcaaatgttatctgcaacaaacaaatatattcatcatttttactactataccatacctg

SEQ ID NO: 19 - DNA, 121 bp, Pisum sativum
  misc_feature 1..121: note = Description of sequence: Oligonucleotide, marker at position 66543611 on pea chromosome 2.
  source 1..121: mol_type = genomic DNA
  variation 61: note = site of single nucleotide polymorphism (SNP)
tagctcaaatgacaaagcagcatctttagtcgtgattccagaaaccaatggacacatgtctgagagtattacagaaaatcctttttgctgcagaaaggaaaatcaaagacacattgttaga

SEQ ID NO: 20 - DNA, 121 bp, Pisum sativum
  misc_feature 1..121: note = Description of sequence: Oligonucleotide, marker at position 66543611 on pea chromosome 2.
  source 1..121: mol_type = genomic DNA
  variation 61: note = site of single nucleotide polymorphism (SNP)
tagctcaaatgacaaagcagcatctttagtcgtgattccagaaaccaatggacacatgtccgagagtattacagaaaatcctttttgctgcagaaaggaaaatcaaagacacattgttaga

SEQ ID NO: 21 - DNA, 121 bp, Pisum sativum
  misc_feature 1..121: note = Description of sequence: Oligonucleotide, marker at position 441796315 on pea chromosome 5.
  source 1..121: mol_type = genomic DNA
  variation 61: note = site of single nucleotide polymorphism (SNP)
tatactagcaagtgaatcccctttgtcatcttttgtttgatcactttcctttgtcatgaaattgaatacattagctttagcattgctagctaacacagaaacactactttggacactttga

SEQ ID NO: 22 - DNA, 121 bp, Pisum sativum
  misc_feature 1..121: note = Description of sequence: Oligonucleotide, marker at position 441796315 on pea chromosome 5.
  source 1..121: mol_type = genomic DNA
  variation 61: note = site of single nucleotide polymorphism (SNP)
tatactagcaagtgaatcccctttgtcatcttttgtttgatcactttcctttgtcatgaagttgaatacattagctttagcattgctagctaacacagaaacactactttggacactttga

SEQ ID NO: 23 - DNA, 121 bp, Pisum sativum
  misc_feature 1..121: note = Description of sequence: Oligonucleotide, marker at position 15003653 on pea chromosome 2.
  source 1..121: mol_type = genomic DNA
  variation 61: note = site of single nucleotide polymorphism (SNP)
gcagctatttggcaaaggattcctatgataatacttattgaacgcataagaagcatgatcatgaatagaattcggattgtaacagcttccagcaggttgaattgctgaacaatcagcgcca

SEQ ID NO: 24 - DNA, 121 bp, Pisum sativum
  misc_feature 1..121: note = Description of sequence: Oligonucleotide, marker at position 15003653 on pea chromosome 2.
  source 1..121: mol_type = genomic DNA
  variation 61: note = site of single nucleotide polymorphism (SNP)
gcagctatttggcaaaggattcctatgataatacttattgaacgcataagaagcatgatcgtgaatagaattcggattgtaacagcttccagcaggttgaattgctgaacaatcagcgcca

SEQ ID NO: 25 - DNA, 121 bp, Pisum sativum
  misc_feature 1..121: note = Description of sequence: Oligonucleotide, marker at position 207468014 on pea chromosome 1.
  source 1..121: mol_type = genomic DNA
  variation 61: note = site of single nucleotide polymorphism (SNP)
tacttttctgaagcaactatcatttttgacaacataggatcaagcgggaactctgccatttgtctgcctaccttagttaatttgccaccaacgtttaaagcattttgtgcgtataaaagct

SEQ ID NO: 26 - DNA, 121 bp, Pisum sativum
  misc_feature 1..121: note = Description of sequence: Oligonucleotide, marker at position 207468014 on pea chromosome 1.
  source 1..121: mol_type = genomic DNA
  variation 61: note = site of single nucleotide polymorphism (SNP)
tacttttctgaagcaactatcatttttgacaacataggatcaagcgggaactctgccattcgtctgcctaccttagttaatttgccaccaacgtttaaagcattttgtgcgtataaaagct

SEQ ID NO: 27 - DNA, 121 bp, Pisum sativum
  misc_feature 1..121: note = Description of sequence: Oligonucleotide, marker at position 5776307 on pea chromosome 2.
  source 1..121: mol_type = genomic DNA
  variation 61: note = site of single nucleotide polymorphism (SNP)
atgggctggtattttcagaaggttccttgtggactctccctacatgtggtggccttcaaaccttgtccaggtgtctctattcaggtatttctttattttttctatcattctctttgtgtgt

SEQ ID NO: 28 - DNA, 121 bp, Pisum sativum
  misc_feature 1..121: note = Description of sequence: Oligonucleotide, marker at position 5776307 on pea chromosome 2.
  source 1..121: mol_type = genomic DNA
  variation 61: note = site of single nucleotide polymorphism (SNP)
atgggctggtattttcagaaggttccttgtggactctccctacatgtggtggccttcaaatcttgtccaggtgtctctattcaggtatttctttattttttctatcattctctttgtgtgt

SEQ ID NO: 29 - DNA, 121 bp, Pisum sativum
  misc_feature 1..121: note = Description of sequence: Oligonucleotide, marker at position 31990510 on pea chromosome 2.
  source 1..121: mol_type = genomic DNA
  variation 61: note = site of single nucleotide polymorphism (SNP)
ttgcaggagacaaaaagaaactttagacccaaccaacccactaagagatttagaagtaagttatctgtctttgaacttaatgtttttagatccatgtagtcaaactcaaggtagtcaaact

SEQ ID NO: 30 - DNA, 121 bp, Pisum sativum
  misc_feature 1..121: note = Description of sequence: Oligonucleotide, marker at position 31990510 on pea chromosome 2.
  source 1..121: mol_type = genomic DNA
  variation 61: note = site of single nucleotide polymorphism (SNP)
ttgcaggagacaaaaagaaactttagacccaaccaacccactaagagatttagaagtaagctatctgtctttgaacttaatgtttttagatccatgtagtcaaactcaaggtagtcaaact

SEQ ID NO: 31 - DNA, 121 bp, Pisum sativum
  misc_feature 1..121: note = Description of sequence: Oligonucleotide, marker at position 82642395 on pea chromosome 3.
  source 1..121: mol_type = genomic DNA
  variation 61: note = site of single nucleotide polymorphism (SNP)
ttcaacaatatgaccccgacgtactacaatctcaatggctcgtgaaccaggttcagaaatctcaccagccattttgcgtgaggctttatctatcttgatgaattcacgaaacatctccact

SEQ ID NO: 32 - DNA, 121 bp, Pisum sativum
  misc_feature 1..121: note = Description of sequence: Oligonucleotide, marker at position 82642395 on pea chromosome 3.
  source 1..121: mol_type = genomic DNA
  variation 61: note = site of single nucleotide polymorphism (SNP)
ttcaacaatatgaccccgacgtactacaatctcaatggctcgtgaaccaggttcagaaatttcaccagccattttgcgtgaggctttatctatcttgatgaattcacgaaacatctccact

SEQ ID NO: 33 - DNA, 121 bp, Pisum sativum
  misc_feature 1..121: note = Description of sequence: Oligonucleotide, marker at position 268444221 on pea chromosome 1.
  source 1..121: mol_type = genomic DNA
  variation 61: note = site of single nucleotide polymorphism (SNP)
cttaaaggacatgttcggcaccccaaacgactgtgaactaatatcagcggttgaaagctcggtttcaatgtctcttggagcaccaccgtaatttgacaacaagttttgaagatctttttga

SEQ ID NO: 34 - DNA, 121 bp, Pisum sativum
  misc_feature 1..121: note = Description of sequence: Oligonucleotide, marker at position 268444221 on pea chromosome 1.
  source 1..121: mol_type = genomic DNA
  variation 61: note = site of single nucleotide polymorphism (SNP)
cttaaaggacatgttcggcaccccaaacgactgtgaactaatatcagcggttgaaagctcagtttcaatgtctcttggagcaccaccgtaatttgacaacaagttttgaagatctttttga

SEQ ID NO: 35 - DNA, 121 bp, Pisum sativum
  misc_feature 1..121: note = Description of sequence: Oligonucleotide, marker at position 5723089 on pea chromosome 2.
  source 1..121: mol_type = genomic DNA
  variation 61: note = site of single nucleotide polymorphism (SNP)
aaaataggaaactgcctgtggagacacaggtatattgctctgtagaggtggggttgggcttggggacaccgcctgtgaacttgcagtctgtaatgttgaaagaattgcagagatattaggg

SEQ ID NO: 36 - DNA, 121 bp, Pisum sativum
  misc_feature 1..121: note = Description of sequence: Oligonucleotide, marker at position 5723089 on pea chromosome 2.
  source 1..121: mol_type = genomic DNA
  variation 61: note = site of single nucleotide polymorphism (SNP)
aaaataggaaactgcctgtggagacacaggtatattgctctgtagaggtggggttgggctgggggacaccgcctgtgaacttgcagtctgtaatgttgaaagaattgcagagatattaggg

SEQ ID NO: 37 - DNA, 121 bp, Pisum sativum
  misc_feature 1..121: note = Description of sequence: Oligonucleotide, marker at position 310787199 on pea chromosome 7.
  source 1..121: mol_type = genomic DNA
  variation 61: note = site of single nucleotide polymorphism (SNP)
aagttgctgcttcaaatgtacttcacatatacaagcatttaaaacgacaagaaaactcaacagatagtaattttgtctactgaatgtttaggctctcttcattagtacaaccattatcacc

SEQ ID NO: 38 - DNA, 121 bp, Pisum sativum
  misc_feature 1..121: note = Description of sequence: Oligonucleotide, marker at position 310787199 on pea chromosome 7.
  source 1..121: mol_type = genomic DNA
  variation 61: note = site of single nucleotide polymorphism (SNP)
aagttgctgcttcaaatgtacttcacatatacaagcatttaaaacgacaagaaaactcaatagatagtaattttgtctactgaatgtttaggctctcttcattagtacaaccattatcacc

SEQ ID NO: 39 - DNA, 121 bp, Pisum sativum
  misc_feature 1..121: note = Description of sequence: Oligonucleotide, marker at position 6738374 on pea chromosome 2.
  source 1..121: mol_type = genomic DNA
  variation 61: note = site of single nucleotide polymorphism (SNP)
tattgtgcttgatgtgctgttgatttttcctgaccttttggaaagagggtttaatccaaaaggtgggattgggttggatttgttgatgagtttggatagcactgtgtttttgttcctgttg

SEQ ID NO: 40 - DNA, 121 bp, Pisum sativum
  misc_feature 1..121: note = Description of sequence: Oligonucleotide, marker at position 6738374 on pea chromosome 2.
  source 1..121: mol_type = genomic DNA
  variation 61: note = site of single nucleotide polymorphism (SNP)
tattgtgcttgatgtgctgttgatttttcctgaccttttggaaagagggtttaatccaaagggtgggattgggttggatttgttgatgagtttggatagcactgtgtttttgttcctgttg

SEQ ID NO: 41 - DNA, 121 bp, Pisum sativum
  misc_feature 1..121: note = Description of sequence: Oligonucleotide, marker at position 125331766 on pea chromosome 4.
  source 1..121: mol_type = genomic DNA
  variation 61: note = site of single nucleotide polymorphism (SNP)
acaatattgtgatgataatggagcatatgtggtggtgacagactatggtgagggagatagaactgatttcatattgagtccacgtgccttttcaagattggggcgcaatgcagttacatca

SEQ ID NO: 42 - DNA, 121 bp, Pisum sativum
  misc_feature 1..121: note = Description of sequence: Oligonucleotide, marker at position 125331766 on pea chromosome 4.
  source 1..121: mol_type = genomic DNA
  variation 61: note = site of single nucleotide polymorphism (SNP)
acaatattgtgatgataatggagcatatgtggtggtgacagactatggtgagggagataggactgatttcatattgagtccacgtgccttttcaagattggggcgcaatgcagttacatca

SEQ ID NO: 43 - DNA, 121 bp, Pisum sativum
  misc_feature 1..121: note = Description of sequence: Oligonucleotide, marker 038887_23884_703 in pea linkage group 4 (LG4) on chromosome 4.
  source 1..121: mol_type = genomic DNA
  variation 61: note = site of single nucleotide polymorphism (SNP)
ctttcttctgtatttccttcttttctttttcctggccaccaaacagcaggttcatatttctcaggaaacttttcaagcataacacctaataaaggaagaggataagctttatcaagagcca

SEQ ID NO: 44 - DNA, 121 bp, Pisum sativum
  misc_feature 1..121: note = Description of sequence: Oligonucleotide, marker 038887_23884_703 in pea linkage group 4 (LG4) on chromosome 4.
  source 1..121: mol_type = genomic DNA
  variation 61: note = site of single nucleotide polymorphism (SNP)
ctttcttctgtatttccttcttttctttttcctggccaccaaacagcaggttcatatttcgcaggaaacttttcaagcataacacctaataaaggaagaggataagctttatcaagagcca

SEQ ID NO: 45 - DNA, 121 bp, Pisum sativum
  misc_feature 1..121: note = Description of sequence: Oligonucleotide, marker 017135_10651_5316 in pea linkage group 5 (LG5) on chromosome 3.
  source 1..121: mol_type = genomic DNA
  variation 61: note = site of single nucleotide polymorphism (SNP)
tgagatcacagttactcaacatacaacttaaatgaaatataacgaattagcataaaactcaagaggagggcatacatcttcaccaattgaaacagcttcagggaagagcccgtgaatgaga

SEQ ID NO: 46 - DNA, 121 bp, Pisum sativum
  misc_feature 1..121: note = Description of sequence: Oligonucleotide, marker 017135_10651_5316 in pea linkage group 5 (LG5) on chromosome 3.
  source 1..121: mol_type = genomic DNA
  variation 61: note = site of single nucleotide polymorphism (SNP)
tgagatcacagttactcaacatacaacttaaatgaaatataacgaattagcataaaactcgagaggagggcatacatcttcaccaattgaaacagcttcagggaagagcccgtgaatgaga

SEQ ID NO: 47 - DNA, 121 bp, Pisum sativum
  misc_feature 1..121: note = Description of sequence: Oligonucleotide, marker 050373_32960_3169 in pea linkage group 3 (LG3) on chromosome 5.
  source 1..121: mol_type = genomic DNA
  variation 61: note = site of single nucleotide polymorphism (SNP)
gtttacataagattagaatgaattgatcactactatacagttttgagaaatgaaatacacaaggaatgcgttatgtacgcagaacagggaaagggaatcaagaatcggtagtggaatcgat

SEQ ID NO: 48 - DNA, 121 bp, Pisum sativum
  misc_feature 1..121: note = Description of sequence: Oligonucleotide, marker 050373_32960_3169 in pea linkage group 3 (LG3) on chromosome 5.
  source 1..121: mol_type = genomic DNA
  variation 61: note = site of single nucleotide polymorphism (SNP)
gtttacataagattagaatgaattgatcactactatacagttttgagaaatgaaatacacgaggaatgcgttatgtacgcagaacagggaaagggaatcaagaatcggtagtggaatcgat

SEQ ID NO: 49 - DNA, 121 bp, Pisum sativum
  misc_feature 1..121: note = Description of sequence: Oligonucleotide, marker 029308_17474_1688 in pea linkage group 1 (LG1) on chromosome 2.
  source 1..121: mol_type = genomic DNA
  variation 61: note = site of single nucleotide polymorphism (SNP)
ttttttggttcttctatagacatattcaactagtttgtttgcatccatggttcctgtcactgttacttttcctgtgctaaactccgtcactgcggtttgaactcctacaataatccataca

SEQ ID NO: 50 - DNA, 121 bp, Pisum sativum
  misc_feature 1..121: note = Description of sequence: Oligonucleotide, marker 029308_17474_1688 in pea linkage group 1 (LG1) on chromosome 2.
  source 1..121: mol_type = genomic DNA
  variation 61: note = site of single nucleotide polymorphism (SNP)
ttttttggttcttctatagacatattcaactagtttgtttgcatccatggttcctgtcacggttacttttcctgtgctaaactccgtcactgcggtttgaactcctacaataatccataca

SEQ ID NO: 51 - DNA, 121 bp, Pisum sativum
  misc_feature 1..121: note = Description of sequence: Oligonucleotide, marker 044073_28004_2765 in pea linkage group 5 (LG5) on chromosome 3.
  source 1..121: mol_type = genomic DNA
  variation 61: note = site of single nucleotide polymorphism (SNP)
ataccatgcaggattagctgcagcaaggacagcagtccttgcattcagtgatgtagtgataccagccttggcaatgctaacagtctgttgttccataacttcatgtatagatgtacgatca

SEQ ID NO: 52 - DNA, 121 bp, Pisum sativum
  misc_feature 1..121: note = Description of sequence: Oligonucleotide, marker 044073_28004_2765 in pea linkage group 5 (LG5) on chromosome 3.
  source 1..121: mol_type = genomic DNA
  variation 61: note = site of single nucleotide polymorphism (SNP)
ataccatgcaggattagctgcagcaaggacagcagtccttgcattcagtgatgtagtgatcccagccttggcaatgctaacagtctgttgttccataacttcatgtatagatgtacgatca

SEQ ID NO: 53 - DNA, 102 bp, Pisum sativum
  misc_feature 1..102: note = Description of sequence: Oligonucleotide, marker 07_32684348 in pea linkage group 2 (LG2) on chromosome 6.
  source 1..102: mol_type = genomic DNA
  variation 51: note = site of single nucleotide polymorphism (SNP)
gtccctaatgctgcttatgctggtggtggcccaaggagttcatggcccgcacaggctccctctggctatggctctatgggttatggaaacactgctccttgg

SEQ ID NO: 54 - DNA, 102 bp, Pisum sativum
  misc_feature 1..102: note = Description of sequence: Oligonucleotide, marker 07_32684348 in pea linkage group 2 (LG2) on chromosome 6.
  source 1..102: mol_type = genomic DNA
  variation 51: note = site of single nucleotide polymorphism (SNP)
gtccctaatgctgcttatgctggtggtggcccaaggagttcatggcccgctcaggctccctctggctatggctctatgggttatggaaacactgctccttgg

SEQ ID NO: 55 - DNA, 121 bp, Pisum sativum
  misc_feature 1..121: note = Description of sequence: Oligonucleotide, marker 42662_26712_871 in pea linkage group 1 (LG1) on chromosome 2.
  source 1..121: mol_type = genomic DNA
  variation 61: note = site of single nucleotide polymorphism (SNP)
aggtggtgtttctgttttgtgttctttacttggtccttttacttcatatgctgttggttctgaagttattggtattcttgttagtttgacacttgattctgaatccaaaaagaatcttatg

SEQ ID NO: 56 - DNA, 121 bp, Pisum sativum
  misc_feature 1..121: note = Description of sequence: Oligonucleotide, marker 42662_26712_871 in pea linkage group 1 (LG1) on chromosome 2.
  source 1..121: mol_type = genomic DNA
  variation 61: note = site of single nucleotide polymorphism (SNP)
aggtggtgtttctgttttgtgttctttacttggtccttttacttcatatgctgttggttccgaagttattggtattcttgttagtttgacacttgattctgaatccaaaaagaatcttatg

SEQ ID NO: 57 - DNA, 121 bp, Pisum sativum
  misc_feature 1..121: note = Description of sequence: Oligonucleotide, marker 044504_28363_461 in pea linkage group 6 (LG6) on chromosome 1.
  source 1..121: mol_type = genomic DNA
  variation 61: note = site of single nucleotide polymorphism (SNP)
acatataatagcacgtcgaagatcttcatcgtccttactacagagcacttgcacatattgtataaggtttggaaacatctctttttccgttgttgatgacaacggaaaaagagacttttgt

SEQ ID NO: 58 - DNA, 121 bp, Pisum sativum
  misc_feature 1..121: note = Description of sequence: Oligonucleotide, marker 044504_28363_461 in pea linkage group 6 (LG6) on chromosome 1.
  source 1..121: mol_type = genomic DNA
  variation 61: note = site of single nucleotide polymorphism (SNP)
acatataatagcacgtcgaagatcttcatcgtccttactacagagcacttgcacatattgcataaggtttggaaacatctctttttccgttgttgatgacaacggaaaaagagacttttgt

SEQ ID NO: 59 - DNA, 122 bp, Pisum sativum
  misc_feature 1..122: note = Description of sequence: Oligonucleotide, marker ER1 in pea linkage group 6 (LG6) on chromosome 1.
  source 1..122: mol_type = genomic DNA
  variation 55: note = site of single nucleotide polymorphism (SNP)
ggtttgcaagggacacaacatttggaagaaggcacttgagcatgtgggctcagtcacctattttgttatggattgtaagggaacttttgttacataaaattaatcatacacattaattaaat

SEQ ID NO: 60 - DNA, 122 bp, Pisum sativum
  misc_feature 1..122: note = Description of sequence: Oligonucleotide, marker ER1 in pea linkage group 6 (LG6) on chromosome 1.
  source 1..122: mol_type = genomic DNA
  variation 55: note = site of single nucleotide polymorphism (SNP)
ggtttgcaagggacacaacatttggaagaaggcacttgagcatgtgggctcagtgacctattttgttatggattgtaagggaacttttgttacataaaattaatcatacacattaattaaat

SEQ ID NO: 61 - DNA, 121 bp, Pisum sativum
  misc_feature 1..121: note = Description of sequence: Oligonucleotide, marker 044835_28587_1878 in pea linkage group 1 (LG1) on chromosome 2.
  source 1..121: mol_type = genomic DNA
  variation 61: note = site of single nucleotide polymorphism (SNP)
tacatcagtttgagaaagttacagcagaactcacaactcaagaagaaacttgcaatttgttatatcaaccggaatttcgccaacgaggtttaagttgctcaaatccagcaattcaagcagc

SEQ ID NO: 62 - DNA, 121 bp, Pisum sativum
  misc_feature 1..121: note = Description of sequence: Oligonucleotide, marker 044835_28587_1878 in pea linkage group 1 (LG1) on chromosome 2.
  source 1..121: mol_type = genomic DNA
  variation 61: note = site of single nucleotide polymorphism (SNP)
tacatcagtttgagaaagttacagcagaactcacaactcaagaagaaacttgcaatttgtgatatcaaccggaatttcgccaacgaggtttaagttgctcaaatccagcaattcaagcagc

SEQ ID NO: 63 - DNA, 121 bp, Pisum sativum
  misc_feature 1..121: note = Description of sequence: Oligonucleotide, marker 044855_28602_1561 in pea linkage group 1 (LG1) on chromosome 2.
  source 1..121: mol_type = genomic DNA
  variation 61: note = site of single nucleotide polymorphism (SNP)
cattacctcacttgaccaagccttcaaccaagcaaagaagcgtagtcaaaaagtttgtggagttataatatcaaacccttcaaaccctaccggaaaattcttaaatcgggaaacactactt

SEQ ID NO: 64 - DNA, 121 bp, Pisum sativum
  misc_feature 1..121: note = Description of sequence: Oligonucleotide, marker 044855_28602_1561 in pea linkage group 1 (LG1) on chromosome 2.
  source 1..121: mol_type = genomic DNA
  variation 61: note = site of single nucleotide polymorphism (SNP)
cattacctcacttgaccaagccttcaaccaagcaaagaagcgtagtcaaaaagtttgtggggttataatatcaaacccttcaaaccctaccggaaaattcttaaatcgggaaacactactt

(19) United States
(12) Patent Application Publication
(10) Pub. No.: US 2026/0033448 A1
(43) Pub. Date: Feb. 5, 2026

(54) NEUTRALLY TASTING, HIGH PROTEIN AND LOW FAT PEA VARIETIES

(71) Applicant: EQUI-nom Ltd., Kibbutz Givat Brenner (IL)

(72) Inventors: Omri LIFSHITZ, Kfar Bilu (IL); Sigal MEIROVITCH, Tel-Aviv (IL); Nicholas RUTLEY, Hod HaSharon (IL); Menachem SKLARZ, Beer Sheva (IL); Noa BAR LISS, Rehovot (IL); Itay ZEMACH, Rehovot (IL); Avichai AMRAD, Kfar Bilu (IL); Noa PALEVSKY, Rehovot (IL); Gil SHALEV, Ramot Meir (IL)

(73) Assignee: EQUI-nom Ltd., Kibbutz Givat Brenner (IL)

(21) Appl. No.: 19/363,823

(22) Filed: Oct. 21, 2025

Related U.S. Application Data
(63) Continuation of application No. PCT/IL2024/050804, filed on Aug. 8, 2024.
(63) Continuation-in-part of application No. 17/630,126, now abandoned.
(60) Provisional application No. 63/518,757, filed on Aug. 10, 2023.

(30) Foreign Application Priority Data
Aug. 21, 2020 (CA) 3090792

(51) Int. Cl. A01H 6/54; A01H 5/10; A23C 11/06; A23J 3/14; A23J 3/22

(52) U.S. Cl. CPC A01H 6/546; A01H 5/10; A23C 11/06; A23J 3/14; A23J 3/227

(57) ABSTRACT

Pea plants or part(s) thereof, as well as products such as commodity whole or split grains, pea protein concentrates (PPC), dairy analogues or protein-fortified beverages and meat analogues or texturized pea protein prepared therefrom, which have high protein content, low fat content and neutral taste are provided. The pea plans, part(s) and products are characterized by specific sets of genetic markers that correspond to identified quantitative trait loci (QTLs). Phenotypic and genotypic analysis of many pea varieties was performed to derive the markers for these phenotypic traits, and a breeding simulation was used to identify the most common and most stable markers. Following verification of trait stability over several generations, markers and marker cassettes were defined as being uniquely present in the developed pea lines. The resulting pea lines can be used to provide products with pea-based protein and low or no off flavors.

### CROSS REFERENCE TO RELATED APPLICATIONS

This application is a Continuation of PCT International Application No. PCT/IL2024/050804, filed Aug. 8, 2024, which claims priority from U.S. Provisional Patent Application No. 63/518,757, filed Aug. 10, 2023, both of which are incorporated by reference in their entirety.

This application is also a Continuation-in-Part of U.S. application Ser. No. 17/630,126 filed Jan. 25, 2022, which is a U.S. National Phase Application of PCT International Application No. PCT/IL2021/051020, filed Aug. 19, 2021, which claims priority from Canadian Patent Application No. 3,090,792, filed Aug. 21, 2020, all of which are incorporated by reference in their entirety.

### SEQUENCE LISTING STATEMENT

A Sequence Listing conforming to the rules of WIPO Standard ST.26 is hereby incorporated by reference. Said Sequence Listing has been filed as an electronic document via Patent Center encoded as XML in UTF-8 text and is hereby incorporated by reference in its entirety. The electronic document, created on Oct. 20, 2025, is entitled “P-628198-US_ST26.xml”, and is 116,557 bytes in size.

### BACKGROUND OF THE INVENTION

### 1. Technical Field

The present invention relates to the field of pea varieties for multiple uses, such as pea concentrates, dairy products and meat replacements, and more particularly, to computer-assisted classically-bred varieties with neutral taste.

### 2. Discussion of Related Art

Pea (Pisum sativum) is a cool season legume grown worldwide as a source of protein both for human food and animal feed. Economically, legumes represent the second most important family of crop plants, and dry pea currently ranks second only to common bean as the most widely grown grain legume in the world. Its primary production is in temperate regions. In 2018, its global production was 34.7 M tons.

Although pea is considered to be one of the world's oldest domesticated crops, classical breeding methodology attempts done in order to increase protein level encountered some obstacles due to inferior agronomical traits such as low yield potential. Commercial dry pea varieties, which currently grown in France and Canada, have an average protein content of about 22%. The plant protein market is constantly challenged by the growing worldwide demand for non-GMO (genetically modified organisms) plant-based protein.

Dry fractionation of pulse proteins has been used as a sustainable method of plant protein extraction due to reduced use of energy and water (see, e.g., Moeller et al. 2021, From raw material to mildly refined ingredient—Linking structure to composition to understand fractionation processes, Journal of Food Engineering 291, 110321). However, since commercial pea concentrates typically have a distinct “beany” flavor (see, e.g., Trindler et al. 2022, Aroma of peas, its constituents and reduction strategies—Effects from breeding to processing, Food Chemistry 376, 131892), their uses in the food industry are limited, and current practice often requires chemical or biological processes to mitigate the undesirable off-flavors.

Tayeh et al. 2015 (Development of two major resources for pea genomics: the GenoPea 13.2 K SNP Array and a high-density, high-resolution consensus genetic map, The Plant Journal, 84(6), pp. 1257-1273) provides extensive data on a very larger set of SNP (single nucleotide polymorphism) markers identified in pea.

### SUMMARY OF THE INVENTION

The following is a simplified summary providing an initial understanding of the invention. The summary does not necessarily identify key elements nor limit the scope of the invention, but merely serves as an introduction to the following description.

One aspect of the present invention provides pea plants or part(s) thereof that have high protein content, low fat content (e.g., at most 2% w/w dry base) and neutral taste, the pea plant(s) comprising a plurality of loci associated with a corresponding plurality quantitative trait loci (QTLs) having a corresponding plurality of nucleic acid genetic markers that are associated with a plurality of phenotypic traits of the pea plant, the phenotypic traits including high yield, high protein, low fat and neutral taste traits, wherein: the phenotypic traits comprise a high protein content of the seeds of at least 29% w/w dry base, a low fat content of the seeds of at most 2% w/w dry base and the neutral taste characterized by moderate to very mild off flavors in a qualitative tasting test and associated with reduced level of volatile compounds associated with off flavors, the plurality of QTLs and corresponding markers comprise at least three QTLs and corresponding markers, of at least one of the following cassettes: (i) cassette 1 comprising: the QTL and marker associated with the increased yield trait comprise QTL 1 with corresponding markers set forth in Seq. IDs 1 or 2, and the pea plant or part thereof is homozygous with respect to Seq. ID 1 or heterozygous at QTL 1, the QTL and marker associated with the low fat content trait comprise QTL 2 with corresponding markers set forth in Seq. IDs 3 or 4, and the pea plant or part thereof is homozygous with respect to Seq. ID 3 or heterozygous at QTL 2, and the QTL and marker associated with the increased yield trait and with a neutral taste trait comprise QTL 3 with corresponding markers set forth in Seq. IDs 5 or 6, and the pea plant or part thereof is homozygous with respect to Seq. ID 5 or heterozygous at QTL 3; (ii) at least one of cassettes 2, 3, 4, 8, 9 or 10 comprising the QTL and marker associated with the neutral taste trait comprise QTL 4 with corresponding markers set forth in Seq. IDs 7 or 8, and the pea plant or part thereof is homozygous with respect to Seq. ID 7 or heterozygous at QTL 4; and/or (iii) at least one of cassettes 5-10 comprising the QTL and marker associated with the neutral taste trait comprise QTL 13 with corresponding markers set forth in Seq. IDs 25 or 26, and the pea plant or part thereof is homozygous with respect to Seq. ID 25 or heterozygous at QTL 13.

One aspect of the present invention provides pea plants or part(s) thereof that include the QTLs of any one or several of cassettes 1-10, as well as various products such as commodity whole or split grains, pea protein concentrate (PPC), dairy analogues or protein-fortified beverages, and/or meat analogues or texturized pea proteins—derived therefrom. The QTLs and cassettes are described in detail herein.

One aspect of the present invention relates to a pea plant or a part thereof that has high protein content, the pea plant comprising: a plurality of loci associated with a corresponding plurality quantitative trait loci (QTLs) having a corresponding plurality of nucleic acid genetic markers that are associated with a plurality of phenotypic traits of the pea plant, wherein: the phenotypic traits comprise a high protein content of the seeds of at least 25% and semi-leafless and powdery mildew resistance, the plurality of QTLs and corresponding markers comprise at least three QTLs and corresponding markers, the QTL and marker associated with the semi-leafless trait comprise QTL 28 with corresponding markers set forth in Seq. IDs 55 or 56, and the pea plant or part thereof is homozygous with respect to Seq. ID 55 or heterozygous at QTL 28.

One aspect of the present invention relates to a pea plant cell comprising: a plurality of loci associated with a corresponding plurality quantitative trait loci (QTLs) having a corresponding plurality of nucleic acid genetic markers that are associated with a plurality of phenotypic traits of a pea plant, a part thereof or pea seeds having high protein content and obtained from said pea plant cell, wherein: the phenotypic traits comprise a high protein content of the seeds of at least 25% and semi-leafless and powdery mildew resistance, the plurality of QTLs and corresponding markers comprise at least three QTLs and corresponding markers, the QTL and marker associated with the semi-leafless trait comprise QTL 28 with corresponding markers set forth in Seq. IDs 55 or 56, and the pea plant cell is homozygous with respect to Seq. ID 55 or heterozygous at QTL 28.

One aspect of the present invention relates to a pea plant or a part thereof that has high protein content, the pea plant comprising: a plurality of loci associated with a corresponding plurality quantitative trait loci (QTLs) having a corresponding plurality of nucleic acid genetic markers that are associated with a plurality of phenotypic traits of the pea plant, wherein: the phenotypic traits comprise a high protein content of the seeds of at least 25% and semi-leafless and powdery mildew resistance, the QTL and marker associated with the high protein trait comprise QTL 22 with corresponding marker set forth in Seq. IDs 43 or 44, the pea plant or part thereof comprise QTL 23 with corresponding marker set forth in Seq. IDs 45 or 46, the QTL and marker associated with the semi-leafless trait comprise QTL 28 with corresponding markers set forth in Seq. IDs 55 or 56, the QTL and marker associated with the powdery mildew resistance trait comprise QTL 30 with corresponding markers set forth in Seq. IDs 59 or 60, the pea plant or part thereof is homozygous with respect to Seq. ID 44 or heterozygous at QTL 22, the pea plant or part thereof is homozygous with respect to Seq. ID 45 or heterozygous at QTL 23, the pea plant or part thereof is homozygous with respect to Seq. ID 55 or heterozygous at QTL 28, and the pea plant or part thereof is homozygous with respect to Seq. ID 60 or heterozygous at QTL 30.

One aspect of the present invention relates to a pea plant or a part thereof that has high protein content, the pea plant comprising: a plurality of loci associated with a corresponding plurality quantitative trait loci (QTLs) having a corresponding plurality of nucleic acid genetic markers that are associated with a plurality of phenotypic traits of the pea plant, wherein: the phenotypic traits comprise a high protein content of the seeds of at least 25% and semi-leafless and powdery mildew resistance, the QTL and marker associated with the high protein trait comprise QTL 22 with corresponding marker set forth in Seq. IDs 43 or 44, the pea plant or part thereof comprise QTL 23 with corresponding marker set forth in Seq. IDs 45 or 46, the QTL and marker associated with the semi-leafless trait comprise QTL 28 with corresponding markers set forth in Seq. IDs 55 or 56, the QTL and marker associated with the powdery mildew resistance trait comprise QTL 29 with corresponding markers set forth in Seq. IDs 57 or 58, the pea plant or part thereof is homozygous with respect to Seq. ID 43 or heterozygous at QTL 22, the pea plant or part thereof is homozygous with respect to Seq. ID 46 or heterozygous at QTL 23, the pea plant or part thereof is homozygous with respect to Seq. ID 55 or heterozygous at QTL 28, and the pea plant or part thereof is homozygous with respect to Seq. ID 57 or heterozygous at QTL 29.

One aspect of the present invention relates to a pea plant or a part thereof that has high protein content, the pea plant comprising: a plurality of loci associated with a corresponding plurality quantitative trait loci (QTLs) having a corresponding plurality of nucleic acid genetic markers that are associated with a plurality of phenotypic traits of the pea plant, wherein: the phenotypic traits comprise a high protein content of the seeds of at least 25% and semi-leafless and powdery mildew resistance, the QTL and marker associated with the high protein trait comprise QTL 24 with corresponding marker set forth in Seq. IDs 47 or 48; and QTL 25 with corresponding marker set forth in Seq. IDs 49 or 50, the QTL and marker associated with the semi-leafless trait comprise QTL 28 with corresponding markers set forth in Seq. IDs 55 or 56, the QTL and marker associated with the powdery mildew resistance trait comprise QTL 29 with corresponding markers set forth in Seq. IDs 57 or 58, the pea plant or part thereof is homozygous with respect to Seq. ID 47 or heterozygous at QTL 24, the pea plant or part thereof is homozygous with respect to Seq. ID 49 or heterozygous at QTL 25, the pea plant or part thereof is homozygous with respect to Seq. ID 55 or heterozygous at QTL 28, and the pea plant or part thereof is homozygous with respect to Seq. ID 57 or heterozygous at QTL 29.

One aspect of the present invention relates to a pea plant or a part thereof that has high protein content, the pea plant comprising: a plurality of loci associated with a corresponding plurality quantitative trait loci (QTLs) having a corresponding plurality of nucleic acid genetic markers that are associated with a plurality of phenotypic traits of the pea plant, wherein: the phenotypic traits comprise a high protein content of the seeds of at least 25% and semi-leafless and powdery mildew resistance, the QTL and marker associated with the high protein trait comprise QTL 26 with corresponding marker set forth in Seq. IDs 51 or 52, the pea plant or part thereof comprise QTL 27 with corresponding marker set forth in Seq. IDs 53 or 54, the QTL and marker associated with the semi-leafless trait comprise QTL 28 with corresponding markers set forth in Seq. IDs 55 or 56, the pea plant or part thereof is homozygous with respect to Seq. ID 51 or heterozygous at QTL 26, the pea plant or part thereof is homozygous with respect to Seq. ID 53 or heterozygous at QTL 27, and the pea plant or part thereof is homozygous with respect to Seq. ID 55 or heterozygous at QTL 28.

Some aspects of the present invention relate to uses of the pea plant or a part thereof, e.g., as textured vegetable products (TVPs), e.g., meat replacements, possibly having above 60% protein, above 65% protein or above 70% protein.

One aspect of the present invention relates to a pea plant cell comprising: a plurality of loci associated with a corresponding plurality quantitative trait loci (QTLs) having a corresponding plurality of nucleic acid genetic markers that are associated with a plurality of phenotypic traits of a pea plant obtained from said pea plant cell, wherein: the phenotypic traits comprise a high protein content of the seeds of at least 25% and semi-leafless and powdery mildew resistance, the QTL and marker associated with the high protein trait comprise QTL 22 with corresponding marker set forth in Seq. IDs 43 or 44, the pea plant cell comprises QTL 23 with corresponding marker set forth in Seq. IDs 45 or 46, the QTL and marker associated with the semi-leafless trait comprise QTL 28 with corresponding markers set forth in Seq. IDs 55 or 56, the QTL and marker associated with the powdery mildew resistance trait comprise QTL 30 with corresponding markers set forth in Seq. IDs 59 or 60, the pea plant cell is homozygous with respect to Seq. ID 44 or heterozygous at QTL 22, the pea plant cell is homozygous with respect to Seq. ID 45 or heterozygous at QTL 23, the pea plant cell is homozygous with respect to Seq. ID 55 or heterozygous at QTL 28, and the pea plant cell is homozygous with respect to Seq. ID 60 or heterozygous at QTL 30.

One aspect of the present invention relates to a pea plant cell comprising: a plurality of loci associated with a corresponding plurality quantitative trait loci (QTLs) having a corresponding plurality of nucleic acid genetic markers that are associated with a plurality of phenotypic traits of a pea plant, a part thereof or pea seeds having high protein content and obtained from said pea plant cell, wherein: the phenotypic traits comprise a high protein content of the seeds of at least 25% and semi-leafless and powdery mildew resistance, the QTL and marker associated with the high protein trait comprise QTL 22 with corresponding marker set forth in Seq. IDs 43 or 44, the pea plant cell comprises QTL 23 with corresponding marker set forth in Seq. IDs 45 or 46, the QTL and marker associated with the semi-leafless trait comprise QTL 28 with corresponding markers set forth in Seq. IDs 55 or 56, the QTL and marker associated with the powdery mildew resistance trait comprise QTL 29 with corresponding markers set forth in Seq. IDs 57 or 58, the pea plant cell is homozygous with respect to Seq. ID 43 or heterozygous at QTL 22, the pea plant cell is homozygous with respect to Seq. ID 46 or heterozygous at QTL 23, the pea plant cell is homozygous with respect to Seq. ID 55 or heterozygous at QTL 28, and the pea plant cell is homozygous with respect to Seq. ID 57 or heterozygous at QTL 29.

One aspect of the present invention relates to a pea plant cell comprising: a plurality of loci associated with a corresponding plurality quantitative trait loci (QTLs) having a corresponding plurality of nucleic acid genetic markers that are associated with a plurality of phenotypic traits of a pea plant, a part thereof or pea seeds having high protein content and obtained from said pea plant cell, wherein: the phenotypic traits comprise a high protein content of the seeds of at least 25% and semi-leafless and powdery mildew resistance, the QTL and marker associated with the high protein trait comprise QTL 24 with corresponding marker set forth in Seq. IDs 47 or 48; and QTL 25 with corresponding marker set forth in Seq. IDs 49 or 50, the QTL and marker associated with the semi-leafless trait comprise QTL 28 with corresponding markers set forth in Seq. IDs 55 or 56, the QTL and marker associated with the powdery mildew resistance trait comprise QTL 29 with corresponding markers set forth in Seq. IDs 57 or 58, the pea plant cell is homozygous with respect to Seq. ID 47 or heterozygous at QTL 24, the pea plant cell is homozygous with respect to Seq. ID 49 or heterozygous at QTL 25, the pea plant cell is homozygous with respect to Seq. ID 55 or heterozygous at QTL 28, and the pea plant cell is homozygous with respect to Seq. ID 57 or heterozygous at QTL 29.

One aspect of the present invention relates to a pea plant cell comprising: a plurality of loci associated with a corresponding plurality quantitative trait loci (QTLs) having a corresponding plurality of nucleic acid genetic markers that are associated with a plurality of phenotypic traits of a pea plant, a part thereof or pea seeds having high protein content and obtained from said pea plant cell, wherein: the phenotypic traits comprise a high protein content of the seeds of at least 25% and semi-leafless and powdery mildew resistance, the QTL and marker associated with the high protein trait comprise QTL 26 with corresponding marker set forth in Seq. IDs 51 or 52, the pea plant cell comprises QTL 27 with corresponding marker set forth in Seq. IDs 53 or 54, the QTL and marker associated with the semi-leafless trait comprise QTL 28 with corresponding markers set forth in Seq. IDs 55 or 56, the pea plant cell is homozygous with respect to Seq. ID 51 or heterozygous at QTL 26, the pea plant cell is homozygous with respect to Seq. ID 53 or heterozygous at QTL 27, and the pea plant cell is homozygous with respect to Seq. ID 55 or heterozygous at QTL 28.

These, additional, and/or other aspects and/or advantages of the present invention are set forth in the detailed description which follows; possibly inferable from the detailed description; and/or learnable by practice of the present invention.

### BRIEF DESCRIPTION OF THE DRAWINGS

For a better understanding of embodiments of the invention and to show how the same may be carried into effect, reference will now be made, purely by way of example, to the accompanying drawings in which like numerals designate corresponding elements or sections throughout. The patent or application file contains at least one drawing executed in color. Copies of this patent or patent application publication with color drawing(s) will be provided by the Office upon request and payment of the necessary fee. In the accompanying drawings:

FIGS. 1A and 1B are high-level schematic illustrations of pea chromosomes with indications of the markers' loci, according to some embodiments of the invention.

FIG. 2 provides a comparison along the taste scale of commercial varieties with disclosed varieties, according to some embodiments of the invention.

FIGS. 3A-3C present experimental results indicating the higher protein content and varying protein composition traits in pea varieties with the disclosed marker cassettes, according to some embodiments of the invention.

FIG. 4 is a high-level schematic illustration of the breeding method, according to some embodiments of the invention.

It will be appreciated that for simplicity and clarity of illustration, elements shown in the figures have not necessarily been drawn to scale. For example, the dimensions of some of the elements may be exaggerated relative to other elements for clarity. Further, where considered appropriate, reference numerals may be repeated among the figures to indicate corresponding or analogous elements.

### DETAILED DESCRIPTION OF THE INVENTION

In the following description, various aspects of the present invention are described. For purposes of explanation, specific configurations and details are set forth in order to provide a thorough understanding of the present invention. However, it will also be apparent to one skilled in the art that the present invention may be practiced without the specific details presented herein. Furthermore, well-known features may have been omitted or simplified in order not to obscure the present invention. With specific reference to the drawings, it is stressed that the particulars shown are by way of example and for purposes of illustrative discussion of the present invention only, and are presented in the cause of providing what is believed to be the most useful and readily understood description of the principles and conceptual aspects of the invention. In this regard, no attempt is made to show structural details of the invention in more detail than is necessary for a fundamental understanding of the invention, the description taken with the drawings making apparent to those skilled in the art how the several forms of the invention may be embodied in practice.

Before at least one embodiment of the invention is explained in detail, it is to be understood that the invention is not limited in its application to the details of construction and the arrangement of the components set forth in the following description or illustrated in the drawings. The invention is applicable to other embodiments that may be practiced or carried out in various ways as well as to combinations of the disclosed embodiments. Also, it is to be understood that the phraseology and terminology employed herein are for the purpose of description and should not be regarded as limiting.

Embodiments of the present invention provide efficient and economical methods and mechanisms for producing neutrally tasting pea varieties for multiple uses such as producing pea concentrates and thereby provide improvements to the additional technological fields of dairy products and meat replacements, as non-limiting examples.

The following application discloses processing methods and appropriate pea varieties for producing improved and new non-GMO (genetically modified organisms) pea protein products. Pea varieties that were developed using computer-assisted breeding programs to have neutral taste (in addition to high protein content and low fat content)—open possibilities for using pea products in many fields from which they were barred due to their typical off-flavor.

Provided are pea plants and parts thereof, which are more neutrally tasting, or have less off-flavor, and/or higher protein than current varieties, as well as their uses and corresponding pea plant cells. Phenotypic and genotypic analysis of many pea varieties was performed to derive markers for neutral taste and other phenotypic traits (e.g., high protein content and low fat content), and a breeding simulation was used to identify the most common and most stable markers. Following verification of trait stability over several generations, markers and marker cassettes were defined as being uniquely present in the developed pea lines. The resulting high-protein low-fat neutrally-tasting pea lines can be used to enhance the range of uses of pea and its products in the food industry. Uses include processing the seeds to yield any of pea protein isolate, pea concentrate, texturized products, milk/meat analogs or milk/meat replacements, commodity whole or split grains, or as animal feed.

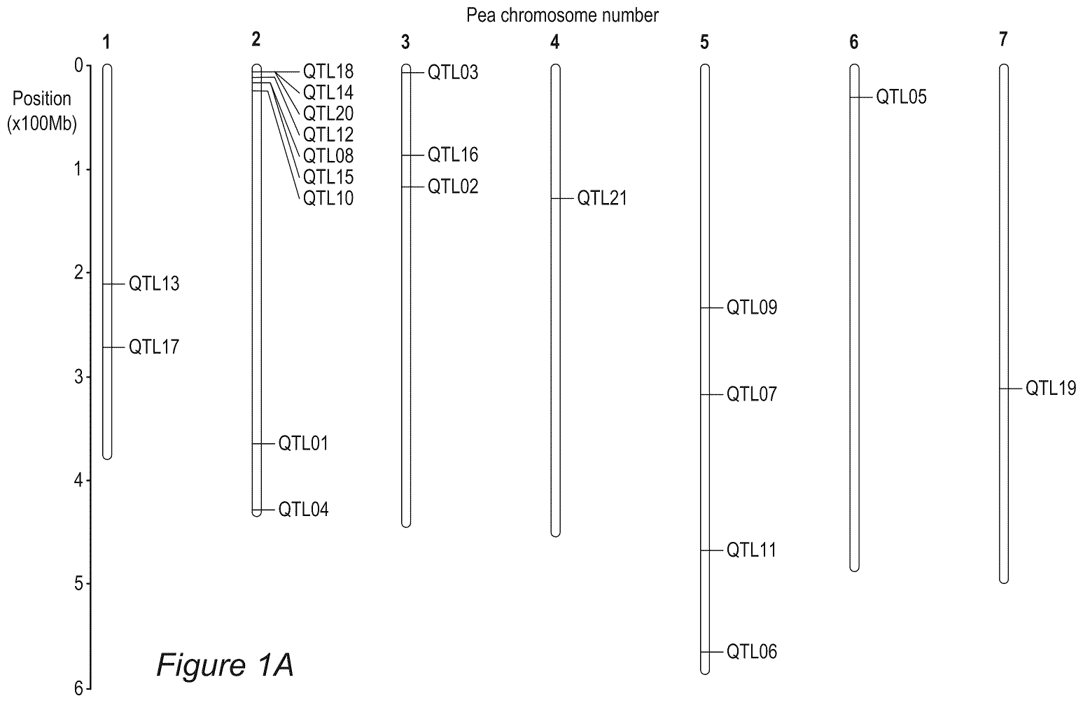

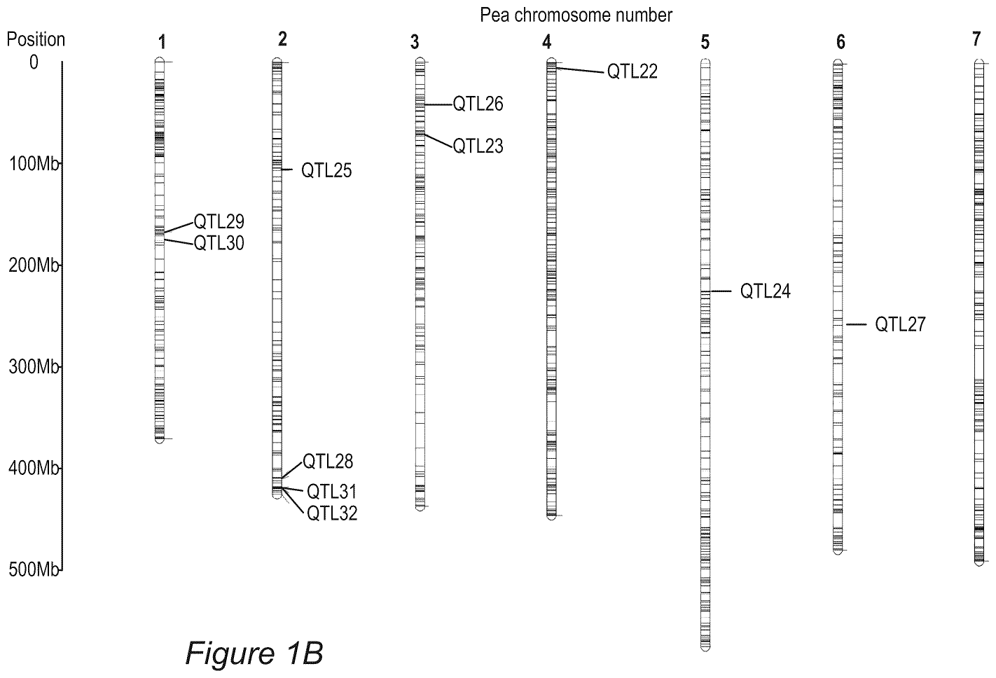

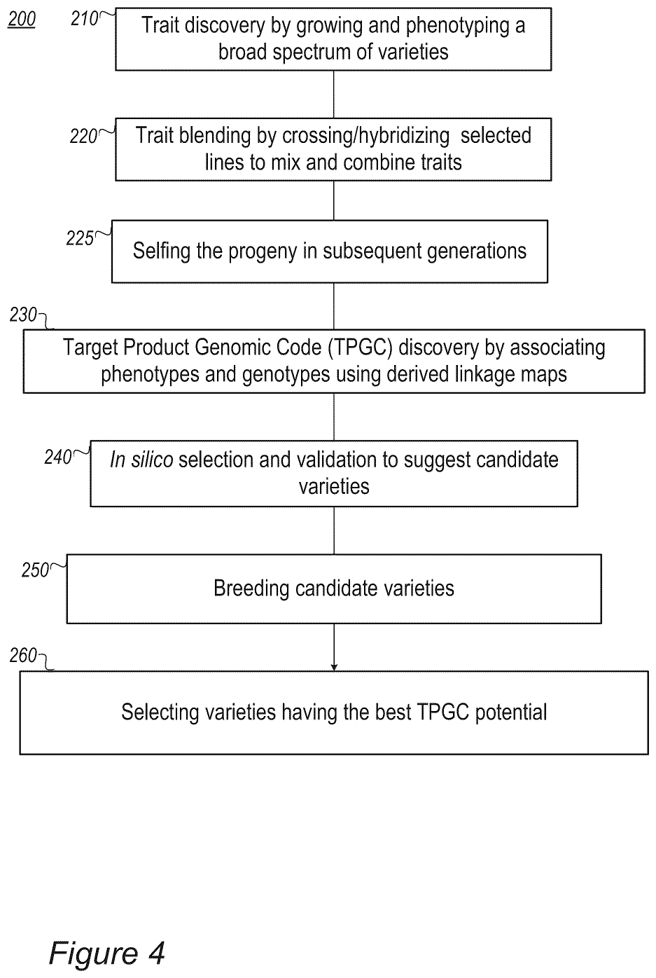

Various embodiments comprise pea plants or part(s) thereof that have a neutral taste and comprise a plurality of loci associated with a corresponding plurality quantitative trait loci (QTLs) having a corresponding plurality of nucleic acid genetic markers that are associated with a plurality of phenotypic traits of the pea plant and/or with a plurality of phenotypic traits of the pea plant. The phenotypic traits comprise high yield, high-protein content, low-fat content, and neutral taste, as well as a high protein content of the seeds of at least 25% and semi-leafless and powdery mildew resistance traits; and the plurality of QTLs and corresponding markers comprise at least two QTLs and corresponding markers—with details provided in FIGS. 1A, 1B and in Table 1 below. The methods used to develop and select the varieties are disclosed with respect to FIG. 4 below.

Various uses include processing the seeds to yield pea protein isolate and/or pea concentrate which provide the pea protein at different levels of concentration and with different amounts of additional compounds. The seeds may be processed into texturized products which may have mechanical properties in addition to their nutritional properties, e.g., texturized products may make a food product more firm or more elastic. The seeds may be processed into meat analogs to provide nutritional properties, chemical characteristics and similar look and feel (e.g., texture, flavor, appearance) as various types of meat, and without off-tastes. The seeds may be processed into commodity whole or split grains, possibly by drying or otherwise modifying the seeds.

FIGS. 1A and 1B are high-level schematic illustrations of pea chromosomes (and linkage groups—LGs) with indications of the markers' loci (QTL number), according to some embodiments of the invention. FIGS. 1A and 1B illustrate schematically the seven pea chromosomes with their banding patterns, and the approximate marker locations indicated along them (see exact data in Table 1). Tables 1-3 provide the derived genetic markers, QTLs, corresponding traits and derived marker cassettes, according to some embodiments of the invention.

Table 1 lists the identified QTLs, markers, associated traits and the significance of correlation between the marker and the trait. Concerning the traits, the numbers of pods and seeds, and seed moisture (in weight %) and weight are agronomic traits related to enhanced yield. The protein content and nutrient fat were measured in weight %, dry basis (protein level refers to weight %, protein content dry basis is calculated by division by the dry weight), and provide nutritional traits that were correlated to the markers (in the direction of higher protein and lower fat contents).

Neutrality of taste was measured by combining a qualitative (sensory) tasting test and a quantitative measurement of volatiles. Volatiles refer to the presence of volatile compounds that affect the aroma, odor and taste, which indicate specifically the off flavors of the pea products. The qualitative tests were performed by trained tasters and the quantitative measurements were carried out by an electronic nose device, as described below.

In the sensory tasting test, each sample was evaluated by multiple tasters which were trained to detect various off flavors according to low concentrate standards including: bitterness-caffeine, sour-citric acid, beany off flavor associated with canned beans, grassy aromas; and mineral water as a neutral taste. The results were analyzed as belonging to one of seven categories:
1—Very neutral taste, no off flavors.
2—Very mild off flavors.
3—Mild off flavors.
4—Moderate off flavors
5—Balanced off flavors.
6—Strong off flavors.
7—Intense off flavors.

Prior art commercial yellow pea varieties and products thereof, such as CDC Meadow, are evaluated at category 7 of the scale above. In contrast, disclosed pea varieties and products thereof are typically rated at categories 2 to 4 of the scale above—exhibiting their superior taste neutrality.

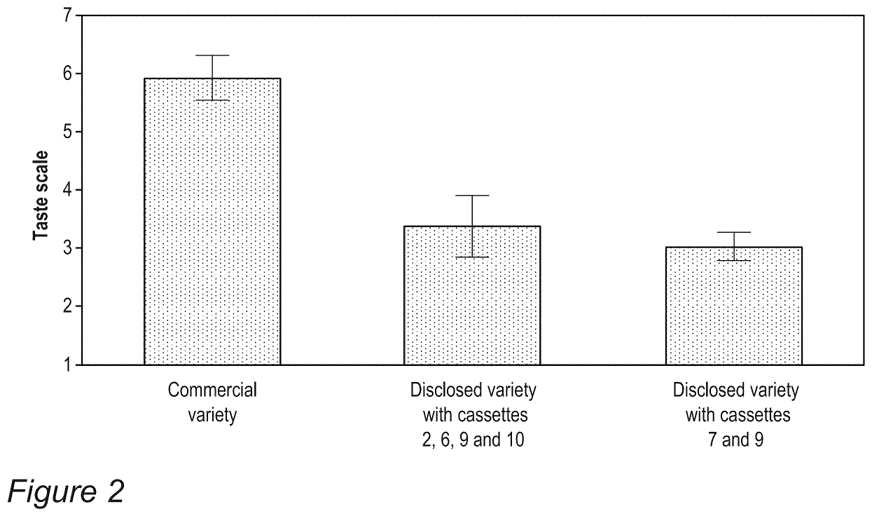

FIG. 2 provides a comparison along the taste scale of commercial varieties (average of 15 taste rankings) with disclosed varieties, according to some embodiments of the invention. The taste comparison has been carried out with milk pea protein concentrate (PPC). Milk PPC was prepared by mixing the PPC (5 wt %) in water, with added taste-neutral stabilizers, emulsifiers (totaling less than 0.1 wt %) and oil (2.8 wt %), according to a specific mixing, heating and cooling protocol used to reach a milk-like texture. One disclosed variety was characterized by markers of cassettes 2, 6, 9 and 10—including QTLs 4, 6, 11, 12, 13, 14, 16, 17, 18, 20 (average of 8 taste rankings) and another disclosed variety was characterized by markers of cassettes 7 and 9 including QTLs 4, 13, 14, 15, 20 (average of 39 taste rankings). The graph indicates that while results for commercial varieties have an average of category 6—Strong off flavors (mean 5.9±0.4), disclosed varieties have averages around category 3—Mild off flavors (mean 3.4±0.5 for QTLs 2, 6, 9 and 10; mean 3.0±0.2 for QTLs 2, 6, 9 and 10, both significantly different from the commercial varieties with p<0.05 in Student's t test), which provides a large and easily noticeable improvement.

In another tasting panel, taste estimations were carried out for the following varieties, with emphasis on the presence of bitter and beany off tastes. Samples were prepared as powder, and in water solution (with a 95:5 ratio of water to concentrate) and two disclosed varieties were compared to commercial concentrates and to commercial de-flavored concentrates (physically processed to reduce off tastes). Of the two tested varieties—one disclosed variety was characterized by markers of cassettes 7 and 8—including QTLs 4, 5, 13, 14, 15, 21 and another disclosed variety was characterized by markers of cassettes 5, 8, 9 and 10 including QTLs 4, 5, 6, 13, 14, 19, 20, 21. Both varieties were ranked three to four ranking steps above the reference de-flavored commercial variety (significantly better than the de-flavored reference in the terms of the test, with four ranking steps being the maximal difference), and five to seven ranking steps above the reference commercial variety (the regular reference commercial variety was graded one to three ranking steps below the reference de-flavored commercial variety—indicating the former being slightly to significantly worse than the latter)—in both powder and water solution format tests. Specifically, the beany and bitter off tastes were shown to be reduced in the disclosed variety compared with the reference de-flavored commercial variety and much reduced in the disclosed variety compared with the reference commercial variety.

In the quantitative tests, the presence of various volatiles was measured using an electronic nose device (e-Nose), which includes gas sensors, configured to detect and identify various volatile organic compounds (VOCs) that are related to odor and taste effects of the peas and pea products. The e-Nose device was used to yield various retention indices for various volatile compounds, following the methodology described in Kakumyan et al. 2009 (Development of a screening system for the evaluation of soybean volatiles, Bioscience, Biotechnology, and Biochemistry, 73(8): 1844-1848). In order to obtain gas chromatography mass spectrometry (GCMS) analyses, retention indices (RI) were calculated from retention time (RT) and data from calibration with 11 hexane standards through the AlphaSoft software (Alpha MOS, Toulouse, France). The RI's were then used to characterize various volatile compounds in the product and correlate them to the qualitative tasting results on the one hand, and to correlate them with the QTL markers listed below on the other hand.

The QTLs relate to these traits in a way that reduces the off flavors in the product. Overall ca. 30 retention indices (RI's), which correspond to multiple volatile compounds, were found to be modified toward taste-neutrality in correlation with the presence of one or more of the disclosed QTLs. These RIs are referred to below, for simplicity, with respect to the neutral taste trait, based on the detected correlations between specific RIs and specific QTLs. It is noted however, that each QTL that is correlated with the neutral taste trait is associated with the presence of one or more volatile compound detected by the corresponding RI. Some exemplary results are provided in Table 5.

Reduction of volatile compounds that contribute to the presence of off tastes—associated with the disclosed QTLs, is considered to correspond to the improved taste neutrality that was shown in the qualitative taste tests, discussed above. Specifically, in various embodiments grassy aromas and off flavors were reduced in varieties characterized by the disclosed QTLs.

TABLE 1
Genetic markers, QTLs and corresponding traits with corresponding phenotypic traits.

| | | Marker | | | QTL | |
|---|---|---|---|---|---|---|
| QTL | Seq ID | (Tayeh et al. 2015) | Chr | Position | P-value | Traits |
| 1 | 1, 2 | 023549_13339_588 | 2 | 363,885,584 | 1.7 · 10⁻⁷ | Number of pods in |
| | | | | | | node |
| 2 | 3, 4 | 001090_925_903 | 3 | 113,590,538 | 7.7 · 10⁻¹⁶ | Nutrient Fat |
| 3 | 5, 6 | 044818_28571_8974 | 3 | 3,563,293 | 1.4 · 10⁻⁵ | Average pods per |
| | | | | | | node, Average seeds |
| | | | | | | per pod, Neutral taste |
| 4 | 7, 8 | 054378_35900_101 | 2 | 427,407,003 | 3.7 · 10⁻⁵ | Neutral taste |
| 5 | 9, 10 | 014081_9600_50 | 6 | 27,314,266 | 0.055 | Protein content (SEQ |
| | | | | | | ID 9), Number of |
| | | | | | | seeds (SEQ ID 10) |
| 6 | 11, 12 | 035862_21021_1237 | 5 | 562,131,030 | 8.7 · 10⁻¹² | Neutral taste |
| 7 | 13, 14 | 043099_27128_757 | 5 | 314,710,599 | 0.0078 | Neutral taste |
| 8 | 15, 16 | 002089_1697_1877 | 2 | 25,529,885 | 4.7 · 10⁻¹⁰⁶ | Neutral taste |
| 9 | 17, 18 | 042929_26966_2757 | 5 | 230,113,430 | 1.5 · 10⁻¹² | Neutral taste |
| 10 | 19, 20 | 005219_3953_563 | 2 | 66,543,611 | 2.0 · 10⁻⁹ | Neutral taste |
| 11 | 21, 22 | 050337_32926_641 | 5 | 441,796,315 | 1.2 · 10⁻⁵ | Neutral taste |
| 12 | 23, 24 | 039413_24354_151 | 2 | 15,003,653 | 1.9 · 10⁻⁶ | Neutral taste |
| 13 | 25, 26 | 000064_54_910 | 1 | 207,468,014 | 0.00094 | Neutral taste |
| 14 | 27, 28 | 042632_26683_1732 | 2 | 5,776,307 | 1.9 · 10⁻²³ | Protein dry basis, |
| | | | | | | Seed moisture |
| 15 | 29, 30 | 051804_34252_2030 | 2 | 31,990,510 | 1.4 · 10⁻¹² | Neutral taste |
| 16 | 31, 32 | 000103_89_2052 | 3 | 82,642,395 | 1.6 · 10⁻⁶ | Neutral taste |
| 17 | 33, 34 | 016931_10524_1358 | 1 | 268,444,221 | 0.13 | Number of seeds per |
| | | | | | | pod |
| 18 | 35, 36 | 042574_26627_1831 | 2 | 5,723,089 | 5.3 · 10⁻⁵ | Neutral taste |
| 19 | 37, 38 | 009756_6539_220 | 7 | 310,787,199 | 0.00060 | Protein dry basis, |
| | | | | | | Protein level |
| 20 | 39, 40 | 001693_1402_686 | 2 | 6,738,374 | 3.3 · 10⁻²⁶ | Protein dry basis, |
| | | | | | | One seed weight |
| 21 | 41, 42 | 039113_24090_857 | 4 | 125,331,766 | 2.6 · 10⁻⁵ | Neutral taste |
| 22 | 43, 44 | 038887_23884_703 | 4 | 6445332 | 0.033 | Protein content |
| 23 | 45, 46 | 017135_10651_5316 | 3 | 71669603 | 0.034 | Protein component |
| 24 | 47, 48 | 050373_32960_3169 | 5 | 225883023 | 0.018 | Protein content |
| 25 | 49, 50 | 029308_17474_1688 | 2 | 106604303 | 0.001 | Protein content |
| 26 | 51, 52 | 044073_28004_2765 | 3 | 44420741 | 0.001 | Protein content |
| 27 | 53, 54 | 07_32684348¹ | 6 | 259389351 | 0.013 | Protein component |
| 28 | 55, 56 | 42662_26712_871 | 2 | 410200645 | Qualitative | Semi-Leafless |
| 29 | 57, 58 | 044504_28363_461 | 1 | 167946502 | markers | Powdery mildew |
| 30 | 59, 60 | ER1¹ | 1 | 175515672 | | resistance |
| 31 | 61, 62 | 044835_28587_1878 | 2 | 419557580 | | Yellow cotyledon² |
| 32 | 63, 64 | 044855_28602_1561 | 2 | 419560368 | | |

¹The markers 07_32684348 was derived from independent RNA sequence analysis, ER1 is provided by Humphry et al. 2011.
²QTLs 31 and 32 for the yellow cotyledon trait are optional.

Table 2 lists the QTLs and markers with respect to their states and their combinations into marker cassettes. Concerning the state of the QTLs in the disclosed varieties, it is noted that typical crop varieties are bred to yield homozygous trait loci, so that in certain embodiments, one or more of the markers is in its homozygous state, all markers in one or more cassettes are in their homozygous state and/or all markers for the respective variety are in their homozygous state (referring to markers associated with the respective QTLs). However, in various embodiments, one or more markers may be in its heterozygous state, with the variety still providing improved agricultural, nutritional and/or taste-neutrality (qualitative test results and quantitative volatiles composition) traits. Accordingly, in varieties characterized by respective marker cassettes—one, several or all markers may be homozygous, and one, several or all markers may be heterozygous. The zygosity (homozygous or heterozygous combination) is provided by the respective cassette entries.

TABLE 2
QTLs, their alleles and the marker cassettes.

| | Seq | Allele | | QTLs and Marker states in Cassette | | | |
|---|---|---|---|---|---|---|---|
| QTL | ID | 1 | 2 | 1 | 2 | 3 | 4 |
| 1 | 1, 2 | A | G | AA/AG | | | |
| 2 | 3, 4 | C | T | CC/CT | | | |
| 3 | 5, 6 | T | C | TT/TC | | | |
| 4 | 7, 8 | T | C | | TT/TC | TT/TC | TT/TC |
| 5 | 9, 10 | C | T | | | | CC/CT |
| 6 | 11, 12 | G | A | | GG/GA | | |
| 7 | 13, 14 | C | A | | | | CC/CA |
| 8 | 15, 16 | C | T | | | | CC/CT |
| 9 | 17, 18 | A | G | | | AA/AG | |
| 10 | 19, 20 | T | C | | | TT/TC | |
| 11 | 21, 22 | A | G | | AA/AG | | |
| 12 | 23, 24 | A | G | | AA/AG | | |

| | Seq | Allele | | QTLs and Marker states in Cassette | | | | | |
|---|---|---|---|---|---|---|---|---|---|
| QTL | ID | 1 | 2 | 5 | 6 | 7 | 8 | 9 | 10 |
| 4 | 7, 8 | T | C | | | | TT/TC | TT/TC | TT/TC |
| 5 | 9, 10 | C | T | TT/TC | | | TT/TC | | |
| 6 | 11, 12 | G | A | | | | | | GG/GA |
| 13 | 25, 26 | T | C | TT/TC | TT/TC | TT/TC | TT/TC | TT/TC | TT/TC |
| 14 | 27, 28 | C | T | | | CC/CT | | CC/CT | |
| 15 | 29, 30 | T | C | | | TT/TC | | | |
| 16 | 31, 32 | C | T | | CC/CT | | | | |
| 17 | 33, 34 | G | A | | GG/GA | | | | |
| 18 | 35, 36 | T | G | | TT/TG | | | | |
| 19 | 37, 38 | C | T | CC/CT | | | | | |
| 20 | 39, 40 | A | G | | | | | AA/AG | AA/AG |
| 21 | 41, 42 | A | G | | | | AA/AG | | |

| | Seq | Allele | | QTLs and Marker states in Cassette³ | | | |
|---|---|---|---|---|---|---|---|
| QTL | ID³ | 1 | 2 | 11 | 12 | 13 | 14 |
| 22 | 43, 44 | T | G | GG/GT | TT/TG | | |
| 23 | 45, 46 | A | G | AA/AG | GG/GA | | |
| 24 | 47, 48 | A | G | | | AA/AG | |
| 25 | 49, 50 | T | G | | | TT/TG | |
| 26 | 51, 52 | A | C | | | | AA/AC |
| 27 | 53, 54 | A | T | | | | AA/AT |
| 28 | 55, 56 | T | C | TT/TC | TT/TC | TT/TC | TT/TC |
| 29 | 57, 58 | T | C | | TT/TC | TT/TC | |
| 30 | 59, 60 | C | G | GG/GC | | | |
| 31² | 61, 62 | T | G | TT/TG | TT/TG | TT/TG | TT/TG |
| 32² | 63, 64 | A | G | AA/AG | AA/AG | AA/AG | AA/AG |

³QTLs 31 and 32 are optional for the respective cassettes.

Table 3 summarizes the composition of the disclosed cassettes of QTLs and markers, and indicates two common marker to multiple disclosed cassettes (QTLs 4 and 13).

TABLE 3
Overview of the marker cassettes.

| | QTLs | |
|---|---|---|
| Cassette | Present | Common |

| | | | |
|---|---|---|---|
| 1 | 1, 2, 3 | | |
| 2 | 4, 6, 11, 12 | 4 | |
| 3 | 4, 9, 10 | | |
| 4 | 4, 5, 7, 8 | | |
| 5 | 5, 13, 19 | | 13 |
| 6 | 13, 16, 17, 18 | | |
| 7 | 13, 14, 15 | | |
| 8 | 4, 5, 13, 21 | 4 | |
| 9 | 4, 13, 14, 20 | | |
| 10 | 4, 6, 13, 20 | | |

| | Obligatory (in 11-14) | Optional |
|---|---|---|
| 11 | 22, 23, 28, 30 | 31, 32 |
| 12 | 22, 23, 28, 29 | 31, 32 |
| 13 | 24, 25, 28, 29 | 31, 32 |
| 14 | 26, 27, 28 | 31, 32 |

Table 4 provides the number of disclosed pea varieties that include various combinations of the disclosed marker cassettes, which are not present in world (prior art) varieties. For each marker cassette, Table 4 presents the number of disclosed varieties that include the cassette by itself and in multiple combinations with other cassettes, compared with no world (prior art) varieties that include these combinations (out of 761 checked world varieties). Overall, 937 pea varieties are disclosed, which are distinct from world varieties by the marker cassettes.

TABLE 4
Disclosed pea varieties with the marker cassettes.
Occurrence in the varieties of cassette number:

| | | | | | | | | | | Number of | Number of |
|---|---|---|---|---|---|---|---|---|---|---|---|
| Occurrence in the varieties of cassette number: | | | | | | | | | | disclosed | world |
| 1 | 2 | 3 | 4 | 5 | 6 | 7 | 8 | 9 | 10 | varieties | lines |
| yes | | | | yes | | | | | | 2 | 0 |
| yes | | | | | | yes | yes | | | 1 | 0 |
| yes | | | | | | | | | | 6 | 0 |
| | yes | | | yes | yes | yes | yes | yes | | 1 | 0 |
| | yes | | | yes | yes | | yes | yes | | 4 | 0 |
| | yes | | | yes | yes | | | | | 2 | 0 |
| | yes | | | yes | | | yes | yes | | 16 | 0 |
| | yes | | | yes | | | | | yes | 11 | 0 |
| | yes | | | yes | | | | | | 124 | 0 |
| | | | | yes | yes | yes | yes | yes | | 2 | 0 |
| | | | | yes | yes | | | | | 20 | 0 |
| | | | | yes | | | yes | yes | | 7 | 0 |
| | | | | yes | | | | | yes | 28 | 0 |
| | | | | yes | | yes | | | | 5 | 0 |
| | | yes | | yes | | | | | | 1 | 0 |
| | | | | yes | | | | | | 324 | 0 |
| | yes | yes | | | yes | | yes | yes | yes | 1 | 0 |
| | yes | yes | | | yes | yes | yes | yes | | 2 | 0 |
| | yes | | yes | | yes | yes | yes | yes | | 2 | 0 |
| | yes | | | | yes | yes | yes | yes | | 3 | 0 |
| | yes | yes | | | yes | | yes | yes | | 2 | 0 |
| | yes | | yes | | yes | | yes | yes | | 5 | 0 |
| | yes | | | | yes | | yes | yes | | 13 | 0 |
| | yes | | | | yes | | | | | 2 | 0 |
| | yes | yes | | | | | yes | yes | yes | 1 | 0 |
| | yes | | | | | | yes | yes | yes | 1 | 0 |
| | yes | | | | | yes | yes | yes | | 1 | 0 |
| | yes | yes | yes | | | | yes | yes | | 4 | 0 |
| | yes | | yes | | | | yes | yes | | 2 | 0 |
| | yes | | | | | | yes | yes | | 10 | 0 |
| | yes | yes | | | | | | | yes | 1 | 0 |
| | yes | | | | | | | | yes | 37 | 0 |
| | yes | yes | | | | | | | | 9 | 0 |
| | yes | | yes | | | | | | | 1 | 0 |
| | yes | | | | | | | | | 150 | 0 |
| | | | | | yes | yes | yes | yes | yes | 7 | 0 |
| | | yes | | | yes | | yes | yes | yes | 1 | 0 |
| | | | | | yes | | yes | yes | yes | 48 | 0 |
| | | yes | | | yes | yes | yes | yes | | 1 | 0 |
| | | | yes | | yes | yes | yes | yes | | 3 | 0 |
| | | | | | yes | yes | yes | yes | | 24 | 0 |
| | | yes | yes | | yes | | yes | yes | | 14 | 0 |
| | | yes | | | yes | | yes | yes | | 3 | 0 |
| | | | yes | | yes | | yes | yes | | 4 | 0 |
| | | | | | yes | | yes | yes | | 41 | 0 |
| | | | | | yes | | | | yes | 2 | 0 |
| | | | | | yes | | | | | 15 | 0 |
| | | | | | | yes | yes | yes | yes | 29 | 0 |
| | | | | | | | yes | yes | yes | 99 | 0 |
| | | yes | | | | yes | yes | yes | | 16 | 0 |
| | | | yes | | | yes | yes | yes | | 3 | 0 |
| | | | | | | yes | yes | yes | | 45 | 0 |
| | | yes | yes | | | | yes | yes | | 5 | 0 |
| | | yes | | | | | yes | yes | | 2 | 0 |
| | | | yes | | | | yes | yes | | 4 | 0 |
| | | | | | | | yes | yes | | 125 | 0 |
| | | | | | | | yes | | | 1 | 0 |
| | | yes | | | | | | yes | yes | 1 | 0 |
| | | | | | | | | | yes | 123 | 0 |
| | | | | | | yes | | | | 302 | 0 |
| | | yes | yes | | | | | | | 3 | 0 |
| | | yes | | | | | | | | 33 | 0 |
| | | | yes | | | | | | | 10 | 0 |

| | | |
|---|---|---|
| Of total: | 937 | 761 |

Table 5 provides some of the retention indices (RI's), which correspond to multiple volatile compounds, that were correlated with the presence of one or more of the disclosed QTLs, and indicate the improved taste-neutrality associated with these markers. The inventors are in the process of identifying the putative molecules and respective odors associated with each RI using e-Nose standards that related specific RI values to specific volatile molecules, initial results are presented in Table 5 with respect to some of the RI's (others are to be determined—denoted TBD). These molecules and odors define off flavors which are reduced in varieties that include the respective QTL markers.

TABLE 5
Taste neutrality QTLs and corresponding retention
indices (RI) and odor descriptions.

| | Taste neutrality traits (RI index) - |
|---|---|
| QTL | Putative molecule - Molecule odor description |
| 4 | RI-1247.86 - 2-isobutyl-3-methoxypyrazine - Green peas, peapods. |
| 6 | RI-963.90 - TBD. |
| 7 | RI-948.75 - TBD. |
| 8 | RI-497.61 - Pentane - Fuel. |
| | RI-527.40 - 2-methylpropanal - Floral, fresh, fruity, green. |
| | RI-601.37 - TBD. |
| | RI-615.53 - TBD. |
| | RI-902.47 - Heptanal - Fatty. |
| | RI-1225.67 - Benzyl alcohol - Floral. |
| | RI-1699.54 - TBD. |
| | RI-466.28 - TBD. |
| | RI-512.42 - TBD. |
| | RI-601.36 - TBD. |
| | RI-723.36 - TBD. |
| | RI-805.43 - TBD. |
| | RI-556.42 - TBD. |
| | RI-683.33 - TBD. |
| | RI-802.49 - Hexanal - Grassy, fatty. |
| 9 | RI-683.33 - TBD. |
| 10 | RI-556.42 - TBD. |
| 11 | RI-1103.40 - Nonanal - Fatty, green-like. |
| 12 | RI-842.18 - TBD. |
| | RI-928.53 - TBD. |
| 13 | RI-1058.04_2 - TBD. |
| 15 | RI-683.33 - TBD. |
| | RI-893.46 - TBD. |
| | RI-963.86 - TBD. |
| 16 | RI-790.24 - TBD. |
| 18 | RI-948.75 - TBD. |
| 21 | RI-638.51 - TBD. |

Table 6 provides protein content and composition data for plant varieties with the marker cassettes, according to some embodiments of the invention—compared to control varieties.

TABLE 6
Protein content and composition data for plant varieties with the marker cassettes.

| | | | | | | Protein composition | | |
|---|---|---|---|---|---|---|---|---|
| | | | | Dunnett's test 0.05 | | (average, relative)¹ | | |
| | Protein content (%) | | | Abs(Dif) − | | Vicilin | Vicilin | P-value |
| Plants type (n) | Average | Max | Min | LSD | p-Value | 6 | 5 | (ANOVA) |
| Cassette 11 (5) | 26.52 | 27.18 | 25.75 | 2.442 | <.0001* | 1.7198 | | 0.0021 |
| Cassette 12 (12) | 26.88 | 28.59 | 25.84 | 2.134 | <.0001* | | 1.10018 | 0.025 |
| Cassette 13 (5) | 26.76 | 27.44 | 26.37 | 1.898 | <.0001* | | | |
| Cassette 14 (2) | 26.4 | 26.81 | 25.98 | 1.381 | 0.0002* | | | |
| Control (5) | 23.41 | 24.56 | 22.43 | −1.22 | 1 | 1.19003 | 1.65058 | |

¹Protein composition includes Vicilin traits 6 and 5 relating to QTL23, for cassettes 11 and 12, respectively.

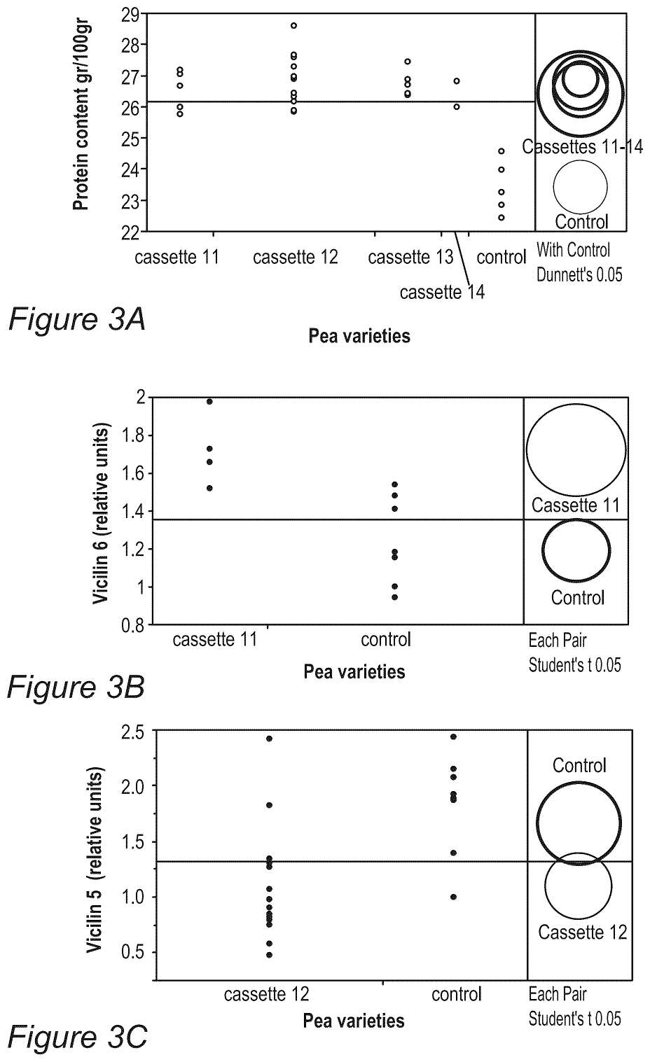

FIGS. 3A-3C present experimental results indicating the higher protein content and varying protein composition traits in pea varieties with the disclosed marker cassettes, according to some embodiments of the invention.

As indicated in Table 6 and illustrated in FIG. 3A, all four cassettes yield average protein content of at least 25% which is significantly distinct from prior art varieties that have lower protein content. FIG. 3A illustrates this significant difference graphically with respect to the multiple lines that were examined for each cassette (denoted by “n” in Table 6).

FIGS. 3B and 3C illustrate schematically the significant differences in protein composition of cassette 11 and cassette 12 varieties, with the former having significantly higher vicilin 6 content than the prior art varieties, and the latter having significantly lower vicilin 5 content than the prior art varieties. It is noted that Vicilin is one of the two major groups of storage proteins (Globulins) present in pea, together with Legumin (and Convicilin) it accounts for about 65-70% of the total protein content in the seed. Vicilin subunits are trimers, that form 6 subunits of 12-50 kDa. Vicilin 5 and 6 have the lowest molecular weight out of the subunits—18 and 16 kDa respectively. All vicilin subunits are characterized by relatively high glycosylation, which contributes to their polarity and thus high water solubility and functionality. Accordingly, the inventors note that differences in protein composition may be related to the nutritional value and the processability of the resulting pea crop.

Disclosed QTLs comprise one or more of QTLs 1 to 32 (see marker names in Table 1) with corresponding pairs of Seq IDs 1-64 that specify the alleles (with respective different SNP—Single Nucleotide Polymorphism—bases) of the respective markers that are linked to QTLs 1-32. It is noted that any of QTLs may be homozygous—having two identical alleles of the same Seq ID; or any of QTLs may be heterozygous having two different alleles with different Seq ID of each pair—as listed in Table 2 and below. Cassettes include groups of QTLs that contribute to the respective traits, as listed in Tables 2, 3 and below. Some of the cassettes include common markers (the main two markers indicated in Table 3) which contribute significantly to the respective traits.

QTL 1, as used herein, refers to a polymorphic genetic locus linked to a genetic marker at position 363885584 on pea chromosome 2. The two alleles of the genetic marker at QTL 1 have the SNP bases “A” or “G”, as set forth respectively in the nucleic acid sequences of SEQ ID NOs 1 and 2. In cassette 1, QTL 1 is homozygous for allele A (SEQ ID NO 1).

SEQ ID NO 1 (SNP base bold):
GATATCTTTTCCAATGACATACTCATCAAGTCAGATGGCAGGACAAGACCTTTCAAA
GAA A TCATGTGAAATTCATCAAGATTCACCAGACTAACACAAAGTAAACAATTATA
CAGAAAAC
SEQ ID NO 2 (SNP base bold):
GATATCTTTTCCAATGACATACTCATCAAGTCAGATGGCAGGACAAGACCTTTCAAA
GAA G TCATGTGAAATTCATCAAGATTCACCAGACTAACACAAAGTAAACAATTATA
CAGAAAAC

QTL 2, as used herein, refers to a polymorphic genetic locus linked to a genetic marker at position 113590538 on pea chromosome 3. The two alleles of the genetic marker at QTL 2 have the SNP bases “C” or “T”, as set forth respectively in the nucleic acid sequences of SEQ ID NOs 3 and 4. In cassette 1, QTL 2 is homozygous for allele C (SEQ ID NO 3).

SEQ ID NO 3 (SNP base bold):
TACTATGGACGATTTCATGAGTTCGTTTTTACTAAACTGTGTTGCTTTTGAACAATGT
TA C AGTGGTTGTTCTATGAAGTATTTCACAACCTATGTAACATTATTGGATTGTTTG
ATAAAT
SEQ ID NO 4 (SNP base bold):
TACTATGGACGATTTCATGAGTTCGTTTTTACTAAACTGTGTTGCTTTTGAACAATGT
TA T AGTGGTTGTTCTATGAAGTATTTCACAACCTATGTAACATTATTGGATTGTTTG
ATAAAT

QTL 3, as used herein, refers to a polymorphic genetic locus linked to a genetic marker at position 3563293 on pea chromosome 3. The two alleles of the genetic marker at QTL 3 have the SNP bases “T” or “C”, as set forth respectively in the nucleic acid sequences of SEQ ID NOs 5 and 6. In cassette 1, QTL 3 is homozygous for allele T (SEQ ID NO 5).

SEQ ID NO 5 (SNP base bold):
CAAAAGTATTTGAGCAGTCATGACTAAAACCTACAGCACAAATAACCAATTGAGTA
GTTG T GGTACAAAAATTCAAAAACACAGGTGGCCGAAGAAGTATAGCTACAAGGT
CAAGAAAATC
SEQ ID NO 6 (SNP base bold):
CAAAAGTATTTGAGCAGTCATGACTAAAACCTACAGCACAAATAACCAATTGAGTA
GTTG C GGTACAAAAATTCAAAAACACAGGTGGCCGAAGAAGTATAGCTACAAGGT
CAAGAAAATC

QTL 4, as used herein, refers to a polymorphic genetic locus linked to a genetic marker at position 427407003 on pea chromosome 2. The two alleles of the genetic marker at QTL 4 have the SNP bases “T” or “C”, as set forth respectively in the nucleic acid sequences of SEQ ID NOs 7 and 8. In cassettes 2-4 and 8-10, QTL 4 is homozygous for allele T (SEQ ID NO 7).

SEQ ID NO 7 (SNP base bold):
CATACCATAGATACATGCTTTAAGAAATGGAACACAAAGATCACCTTAGAAATGATG
TGC T AGCTACAAAGATCACTTTATGATTTGAAGCAGACAGGTAGGAAATGAAACAC
AAAACATA
SEQ ID NO 8 (SNP base bold):
CATACCATAGATACATGCTTTAAGAAATGGAACACAAAGATCACCTTAGAAATGATG
TGC C AGCTACAAAGATCACTTTATGATTTGAAGCAGACAGGTAGGAAATGAAACAC
AAAACATA

QTL 5, as used herein, refers to a polymorphic genetic locus linked to a genetic marker at position 27314266 on pea chromosome 6. The two alleles of the genetic marker at QTL 5 have the SNP bases “C” or “T”, as set forth respectively in the nucleic acid sequences of SEQ ID NOs 9 and 10. In cassette 4, QTL 5 is homozygous for allele C (SEQ ID NO 9), while in cassettes 5 and 8, QTL 5 is homozygous for allele T SEQ ID NO 10).

SEQ ID NO 9 (SNP base bold):
TGTGATGTATCAGTGTGACAAACAGCAACAGCTTTAACTCTATTTCCAT C AACACCT
TCCAATGGCACCGAGTAAACTTTAGTTGTGTCTGTTTTATGACAGTAGAAAAC
SEQ ID NO 10 (SNP base bold):
TGTGATGTATCAGTGTGACAAACAGCAACAGCTTTAACTCTATTTCCAT T AACACCT
TCCAATGGCACCGAGTAAACTTTAGTTGTGTCTGTTTTATGACAGTAGAAAAC

QTL 6, as used herein, refers to a polymorphic genetic locus linked to a genetic marker at position 562131030 on pea chromosome 5. The two alleles of the genetic marker at QTL 6 have the SNP bases “G” or “A”, as set forth respectively in the nucleic acid sequences of SEQ ID NOs 11 and 12. In cassettes 2 and 10, QTL 6 is homozygous for allele G (SEQ ID NO 11).

SEQ ID NO 11 (SNP base bold):
CTTTTCTAAAACTAAGTTCACCAGATTTCCCTTACTTGAAAAATTGGATCTATGTGTT
TC G GATGATTTGAAATCTATTACAATTTATAACCATTGTCTTCAGAGAATTGCTTTA
AAAGGA
SEQ ID NO 12 (SNP base bold):
CTTTTCTAAAACTAAGTTCACCAGATTTCCCTTACTTGAAAAATTGGATCTATGTGTT
TC A GATGATTTGAAATCTATTACAATTTATAACCATTGTCTTCAGAGAATTGCTTTA
AAAGGA

QTL 7, as used herein, refers to a polymorphic genetic locus linked to a genetic marker at position 314710599 on pea chromosome 5. The two alleles of the genetic marker at QTL 7 have the SNP bases “C” or “A”, as set forth respectively in the nucleic acid sequences of SEQ ID NOs 13 and 14. In cassette 4, QTL 7 is homozygous for allele C (SEQ ID NO 13).

SEQ ID NO 13 (SNP base bold):
ACAAACATGCCACTTGGCAGCTAACCCTGTGATTGCTTGTGCTTTGTGTGTTATCTTT
GT C GCACTTCGGAACATGATGGACAACGCAGAAAGAAGCGTCACCGAACACAACT
GCAACAAC
SEQ ID NO 14 (SNP base bold):
ACAAACATGCCACTTGGCAGCTAACCCTGTGATTGCTTGTGCTTTGTGTGTTATCTTT
GT A GCACTTCGGAACATGATGGACAACGCAGAAAGAAGCGTCACCGAACACAACT
GCAACAAC

QTL 8, as used herein, refers to a polymorphic genetic locus linked to a genetic marker at position 25529885 on pea chromosome 2. The two alleles of the genetic marker at QTL 8 have the SNP bases “C” or “T”, as set forth respectively in the nucleic acid sequences of SEQ ID NOs 15 and 16. In cassette 4, QTL 8 is homozygous for allele C (SEQ ID NO 15).

SEQ ID NO 15 (SNP base bold):
CCGAAGCCATTAAAGAGCAAAATTGGGTCAATGATCATGAAGTGTTTGAGTTTGGTT
GGT C TCCGTCTGCATTTGGAAGATGGGAAGAAGAATTTGGATGAAGATGAAGGTTG
GGGAAGAG
SEQ ID NO 16 (SNP base bold):
CCGAAGCCATTAAAGAGCAAAATTGGGTCAATGATCATGAAGTGTTTGAGTTTGGTT
GGT T TCCGTCTGCATTTGGAAGATGGGAAGAAGAATTTGGATGAAGATGAAGGTTG
GGGAAGAG

QTL 9, as used herein, refers to a polymorphic genetic locus linked to a genetic marker at position 230113430 on pea chromosome 5. The two alleles of the genetic marker at QTL 9 have the SNP bases “A” or “G”, as set forth respectively in the nucleic acid sequences of SEQ ID NOs 17 and 18. In cassette 3, QTL 9 is homozygous for allele A (SEQ ID NO 17).

SEQ ID NO 17 (SNP base bold):
CTTTCCTTAAACTCTCTGTCCATTCATCTGGATCAAAACTCATCAACTGTTTCACTGTC
A A CTCAAATGTTATCTGCAACAAACAAATATATTCATCATTTTTACTACTATACCAT
ACCTG
SEQ ID NO 18 (SNP base bold):
CTTTCCTTAAACTCTCTGTCCATTCATCTGGATCAAAACTCATCAACTGTTTCACTGTC
A G CTCAAATGTTATCTGCAACAAACAAATATATTCATCATTTTTACTACTATACCAT
ACCTG

QTL 10, as used herein, refers to a polymorphic genetic locus linked to a genetic marker at position 66543611 on pea chromosome 2. The two alleles of the genetic marker at QTL 10 have the SNP bases “T” or “C”, as set forth respectively in the nucleic acid sequences of SEQ ID NOs 19 and 20. In cassette 3, QTL 10 is homozygous for allele T (SEQ ID NO 19).

SEQ ID NO 19 (SNP base bold):
TAGCTCAAATGACAAAGCAGCATCTTTAGTCGTGATTCCAGAAACCAATGGACACAT
GTC T GAGAGTATTACAGAAAATCCTTTTTGCTGCAGAAAGGAAAATCAAAGACACA
TTGTTAGA
SEQ ID NO 20 (SNP base bold):
TAGCTCAAATGACAAAGCAGCATCTTTAGTCGTGATTCCAGAAACCAATGGACACAT
GTC C GAGAGTATTACAGAAAATCCTTTTTGCTGCAGAAAGGAAAATCAAAGACACA
TTGTTAGA

QTL 11, as used herein, refers to a polymorphic genetic locus linked to a genetic marker at position 441796315 on pea chromosome 5. The two alleles of the genetic marker at QTL 11 have the SNP bases “A” or “G”, as set forth respectively in the nucleic acid sequences of SEQ ID NOs 21 and 22. In cassette 2, QTL 11 is homozygous for allele A (SEQ ID NO 21).

SEQ ID NO 21 (SNP base bold):
TATACTAGCAAGTGAATCCCCTTTGTCATCTTTTGTTTGATCACTTTCCTTTGTCATGA
A A TTGAATACATTAGCTTTAGCATTGCTAGCTAACACAGAAACACTACTTTGGACA
CTTTGA
SEQ ID NO 22 (SNP base bold):
TATACTAGCAAGTGAATCCCCTTTGTCATCTTTTGTTTGATCACTTTCCTTTGTCATGA
A G TTGAATACATTAGCTTTAGCATTGCTAGCTAACACAGAAACACTACTTTGGACA
CTTTGA

QTL 12, as used herein, refers to a polymorphic genetic locus linked to a genetic marker at position 15003653 on pea chromosome 2. The two alleles of the genetic marker at QTL 12 have the SNP bases “A” or “G”, as set forth respectively in the nucleic acid sequences of SEQ ID NOs 23 and 24. In cassette 2, QTL 12 is homozygous for allele A (SEQ ID NO 23).

SEQ ID NO 23 (SNP base bold):
GCAGCTATTTGGCAAAGGATTCCTATGATAATACTTATTGAACGCATAAGAAGCATG
ATC A TGAATAGAATTCGGATTGTAACAGCTTCCAGCAGGTTGAATTGCTGAACAAT
CAGCGCCA
SEQ ID NO 24 (SNP base bold):
GCAGCTATTTGGCAAAGGATTCCTATGATAATACTTATTGAACGCATAAGAAGCATG
ATC G TGAATAGAATTCGGATTGTAACAGCTTCCAGCAGGTTGAATTGCTGAACAAT
CAGCGCCA

QTL 13, as used herein, refers to a polymorphic genetic locus linked to a genetic marker at position 207468014 on pea chromosome 1. The two alleles of the genetic marker at QTL 13 have the SNP bases “T” or “C”, as set forth respectively in the nucleic acid sequences of SEQ ID NOs 25 and 26. In cassettes 5-10, QTL 13 is homozygous for allele T (SEQ ID NO 25).

SEQ ID NO 25 (SNP base bold):
TACTTTTCTGAAGCAACTATCATTTTTGACAACATAGGATCAAGCGGGAACTCTGCC
ATT T GTCTGCCTACCTTAGTTAATTTGCCACCAACGTTTAAAGCATTTTGTGCGTAT
AAAAGCT
SEQ ID NO 26 (SNP base bold):
TACTTTTCTGAAGCAACTATCATTTTTGACAACATAGGATCAAGCGGGAACTCTGCC
ATT C GTCTGCCTACCTTAGTTAATTTGCCACCAACGTTTAAAGCATTTTGTGCGTAT
AAAAGCT

QTL 14, as used herein, refers to a polymorphic genetic locus linked to a genetic marker at position 5776307 on pea chromosome 2. The two alleles of the genetic marker at QTL 14 have the SNP bases “C” or “T”, as set forth respectively in the nucleic acid sequences of SEQ ID NOs 27 and 28. In cassettes 7 and 9, QTL 14 is homozygous for allele C (SEQ ID NO 27).

SEQ ID NO 27 (SNP base bold):
ATGGGCTGGTATTTTCAGAAGGTTCCTTGTGGACTCTCCCTACATGTGGTGGCCTTCA
AA C CTTGTCCAGGTGTCTCTATTCAGGTATTTCTTTATTTTTTCTATCATTCTCTTTGT
GTGT
SEQ ID NO 28 (SNP base bold):
ATGGGCTGGTATTTTCAGAAGGTTCCTTGTGGACTCTCCCTACATGTGGTGGCCTTCA
AA T CTTGTCCAGGTGTCTCTATTCAGGTATTTCTTTATTTTTTCTATCATTCTCTTTGT
GTGT

QTL 15, as used herein, refers to a polymorphic genetic locus linked to a genetic marker at position 31990510 on pea chromosome 2. The two alleles of the genetic marker at QTL 15 have the SNP bases “T” or “C”, as set forth respectively in the nucleic acid sequences of SEQ ID NOs 29 and 30. In cassette 7, QTL 15 is homozygous for allele T (SEQ ID NO 29).

SEQ ID NO 29 (SNP base bold):
TTGCAGGAGACAAAAAGAAACTTTAGACCCAACCAACCCACTAAGAGATTTAGAAG
TAAG T TATCTGTCTTTGAACTTAATGTTTTTAGATCCATGTAGTCAAACTCAAGGTA
GTCAAACT
SEQ ID NO 30 (SNP base bold):
TTGCAGGAGACAAAAAGAAACTTTAGACCCAACCAACCCACTAAGAGATTTAGAAG
TAAG C TATCTGTCTTTGAACTTAATGTTTTTAGATCCATGTAGTCAAACTCAAGGTA
GTCAAACT

QTL 16, as used herein, refers to a polymorphic genetic locus linked to a genetic marker at position 82642395 on pea chromosome 3. The two alleles of the genetic marker at QTL 16 have the SNP bases “C” or “T”, as set forth respectively in the nucleic acid sequences of SEQ ID NOs 31 and 32. In cassette 6, QTL 16 is homozygous for allele C (SEQ ID NO 31).

SEQ ID NO 31 (SNP base bold):
TTCAACAATATGACCCCGACGTACTACAATCTCAATGGCTCGTGAACCAGGTTCAGA
AAT C TCACCAGCCATTTTGCGTGAGGCTTTATCTATCTTGATGAATTCACGAAACAT
CTCCACT
SEQ ID NO 32 (SNP base bold):
TTCAACAATATGACCCCGACGTACTACAATCTCAATGGCTCGTGAACCAGGTTCAGA
AAT T TCACCAGCCATTTTGCGTGAGGCTTTATCTATCTTGATGAATTCACGAAACAT
CTCCACT

QTL 17, as used herein, refers to a polymorphic genetic locus linked to a genetic marker at position 268444221 on pea chromosome 1. The two alleles of the genetic marker at QTL 17 have the SNP bases “G” or “A”, as set forth respectively in the nucleic acid sequences of SEQ ID NOs 33 and 34. In cassette 6, QTL 17 is homozygous for allele G (SEQ ID NO 33).

SEQ ID NO 33 (SNP base bold):
CTTAAAGGACATGTTCGGCACCCCAAACGACTGTGAACTAATATCAGCGGTTGAAAG
CTC G GTTTCAATGTCTCTTGGAGCACCACCGTAATTTGACAACAAGTTTTGAAGATC
TTTTTGA
SEQ ID NO 34 (SNP base bold):
CTTAAAGGACATGTTCGGCACCCCAAACGACTGTGAACTAATATCAGCGGTTGAAAG
CTC A GTTTCAATGTCTCTTGGAGCACCACCGTAATTTGACAACAAGTTTTGAAGATC
TTTTTGA

QTL 18, as used herein, refers to a polymorphic genetic locus linked to a genetic marker at position 5723089 on pea chromosome 2. The two alleles of the genetic marker at QTL 18 have the SNP bases “T” or “G”, as set forth respectively in the nucleic acid sequences of SEQ ID NOs 35 and 36. In cassette 6, QTL 18 is homozygous for allele T (SEQ ID NO 35).

SEQ ID NO 35 (SNP base bold):
AAAATAGGAAACTGCCTGTGGAGACACAGGTATATTGCTCTGTAGAGGTGGGGTTG
GGCT T GGGGACACCGCCTGTGAACTTGCAGTCTGTAATGTTGAAAGAATTGCAGAG
ATATTAGGG
SEQ ID NO 36 (SNP base bold):
AAAATAGGAAACTGCCTGTGGAGACACAGGTATATTGCTCTGTAGAGGTGGGGTTG
GGCT G GGGGACACCGCCTGTGAACTTGCAGTCTGTAATGTTGAAAGAATTGCAGAG
ATATTAGGG

QTL 19, as used herein, refers to a polymorphic genetic locus linked to a genetic marker at position 310787199 on pea chromosome 7. The two alleles of the genetic marker at QTL 19 have the SNP bases “C” or “T”, as set forth respectively in the nucleic acid sequences of SEQ ID NOs 37 and 38. In cassette 5, QTL 19 is homozygous for allele C (SEQ ID NO 37).

SEQ ID NO 37 (SNP base bold):
AAGTTGCTGCTTCAAATGTACTTCACATATACAAGCATTTAAAACGACAAGAAAACT
CAA C AGATAGTAATTTTGTCTACTGAATGTTTAGGCTCTCTTCATTAGTACAACCAT
TATCACC
SEQ ID NO 38 (SNP base bold):
AAGTTGCTGCTTCAAATGTACTTCACATATACAAGCATTTAAAACGACAAGAAAACT
CAA T AGATAGTAATTTTGTCTACTGAATGTTTAGGCTCTCTTCATTAGTACAACCAT
TATCACC

QTL 20, as used herein, refers to a polymorphic genetic locus linked to a genetic marker at position 6738374 on pea chromosome 2. The two alleles of the genetic marker at QTL 20 have the SNP bases “A” or “G”, as set forth respectively in the nucleic acid sequences of SEQ ID NOs 39 and 40. In cassettes 9 and 10, QTL 20 is homozygous for allele A (SEQ ID NO 39).

SEQ ID NO 39 (SNP base bold):
TATTGTGCTTGATGTGCTGTTGATTTTTCCTGACCTTTTGGAAAGAGGGTTTAATCCA
AA A GGTGGGATTGGGTTGGATTTGTTGATGAGTTTGGATAGCACTGTGTTTTTGTTC
CTGTTG
SEQ ID NO 40 (SNP base bold):
TATTGTGCTTGATGTGCTGTTGATTTTTCCTGACCTTTTGGAAAGAGGGTTTAATCCA
AA G GGTGGGATTGGGTTGGATTTGTTGATGAGTTTGGATAGCACTGTGTTTTTGTTC
CTGTTG

QTL 21, as used herein, refers to a polymorphic genetic locus linked to a genetic marker at position 125331766 on pea chromosome 4. The two alleles of the genetic marker at QTL 21 have the SNP bases “A” or “G”, as set forth respectively in the nucleic acid sequences of SEQ ID NOs 41 and 42. In cassette 8, QTL 21 is homozygous for allele A (SEQ ID NO 41).

SEQ ID NO 41 (SNP base bold):
ACAATATTGTGATGATAATGGAGCATATGTGGTGGTGACAGACTATGGTGAGGGAG
ATAG A ACTGATTTCATATTGAGTCCACGTGCCTTTTCAAGATTGGGGCGCAATGCAG
TTACATCA
SEQ ID NO 42 (SNP base bold):
ACAATATTGTGATGATAATGGAGCATATGTGGTGGTGACAGACTATGGTGAGGGAG
ATAG G ACTGATTTCATATTGAGTCCACGTGCCTTTTCAAGATTGGGGCGCAATGCA
GTTACATCA

QTL 22, as used herein, refers to a polymorphic genetic locus linked to genetic marker 038887_23884_703 in pea linkage group 4 (LG4) on chromosome 4. The two alleles of marker 038887_23884_703 at QTL 22 have the SNP bases “T” or “G”, respectively, at position 6445332 of LG4, as set forth, respectively, in the nucleic acid sequences of Seq IDs 43 and 44. In cassette 11, QTL 22 may be homozygous for allele 2 (Seq ID 44) or be heterozygous (Seq IDs 43 and 44); while in cassette 12, QTL 22 may be homozygous for allele 1 (Seq ID 43) or be heterozygous (Seq IDs 43 and 44).

Seq ID No. 43 (SNP base bold):
CTTTCTTCTGTATTTCCTTCTTTTCTTTTTCCTGGCCACCAAACAGCAGGTTCATATTT
CTCAGGAAACTTTTCAAGCATAACACCTAATAAAGGAAGAGGATAAGCTTTATCAA
GAGCCA
Seq ID No. 44 (SNP base bold):
CTTTCTTCTGTATTTCCTTCTTTTCTTTTTCCTGGCCACCAAACAGCAGGTTCATATTT
CGCAGGAAACTTTTCAAGCATAACACCTAATAAAGGAAGAGGATAAGCTTTATCAA
GAGCCA

QTL 23, as used herein, refers to a polymorphic genetic locus linked to genetic marker 017135_10651_5316 in pea linkage group 5 (LG5) on chromosome 3. The two alleles of marker 017135_10651_5316 at QTL 23 have the bases “A” or “G”, respectively, at position 71669603 of LG5, as set forth, respectively, in the nucleic acid sequences of Seq IDs 45 and 46. In cassette 11, QTL 23 may be homozygous for allele 1 (Seq ID 45) or be heterozygous (Seq IDs 45 and 46); while in cassette 12, QTL 23 may be homozygous for allele 2 (Seq ID 46) or be heterozygous (Seq IDs 45 and 46).

Seq ID No. 45 (SNP base bold):
TGAGATCACAGTTACTCAACATACAACTTAAATGAAATATAACGAATTAGCATAAAA
CTCAAGAGGAGGGCATACATCTTCACCAATTGAAACAGCTTCAGGGAAGAGCCCGT
GAATGAGA
Seq ID No. 46 (SNP base bold):
TGAGATCACAGTTACTCAACATACAACTTAAATGAAATATAACGAATTAGCATAAAA
CTCGAGAGGAGGGCATACATCTTCACCAATTGAAACAGCTTCAGGGAAGAGCCCGT
GAATGAGA

QTL 24, as used herein, refers to a polymorphic genetic locus linked to genetic marker 050373_32960_3169 in pea linkage group 3 (LG3) on chromosome 5. The two alleles of marker 050373_32960_3169 at QTL 24 have the bases “A” or “G”, respectively, at position 225883023 of LG3, as set forth, respectively, in the nucleic acid sequences of Seq IDs 47 and 48. In cassette 13, QTL 24 may be homozygous for allele 1 (Seq ID 47) or be heterozygous (Seq IDs 47 and 48).

Seq ID No. 47 (SNP base bold):
GTTTACATAAGATTAGAATGAATTGATCACTACTATACAGTTTTGAGAAATGAAATA
CACAAGGAATGCGTTATGTACGCAGAACAGGGAAAGGGAATCAAGAATCGGTAGTG
GAATCGAT
Seq ID No. 48 (SNP base bold):
GTTTACATAAGATTAGAATGAATTGATCACTACTATACAGTTTTGAGAAATGAAATA
CACGAGGAATGCGTTATGTACGCAGAACAGGGAAAGGGAATCAAGAATCGGTAGTG
GAATCGAT

QTL 25, as used herein, refers to a polymorphic genetic locus linked to genetic marker 029308_17474_1688 in pea linkage group 1 (LG1) on chromosome 2. The two alleles of marker 029308_17474_1688 at QTL 25 have the bases “T” or “G”, respectively, at position 106604303 of LG1, as set forth, respectively, in the nucleic acid sequences of Seq IDs 49 and 50. In cassette 13, QTL 25 may be homozygous for allele 1 (Seq ID 49) or be heterozygous (Seq IDs 49 and 50).

Seq ID No. 49 (SNP base bold):
TTTTTTGGTTCTTCTATAGACATATTCAACTAGTTTGTTTGCATCCATGGTTCCTGTCA
CTGTTACTTTTCCTGTGCTAAACTCCGTCACTGCGGTTTGAACTCCTACAATAATCCA
TACA
Seq ID No. 50 (SNP base bold):
TTTTTTGGTTCTTCTATAGACATATTCAACTAGTTTGTTTGCATCCATGGTTCCTGTCA
CGGTTACTTTTCCTGTGCTAAACTCCGTCACTGCGGTTTGAACTCCTACAATAATCCA
TACA

QTL 26, as used herein, refers to a polymorphic genetic locus linked to genetic marker 044073_28004_2765 in pea linkage group 5 (LG5) on chromosome 3. The two alleles of marker 044073_28004_2765 at QTL 26 have the bases “A” or “C”, respectively, at position 44420741 of LG5, as set forth, respectively, in the nucleic acid sequences of Seq IDs 51 and 52. In cassette 14, QTL 26 may be homozygous for allele 1 (Seq ID 51) or be heterozygous (Seq IDs 51 and 52).

Seq ID No. 51 (SNP base bold):
ATACCATGCAGGATTAGCTGCAGCAAGGACAGCAGTCCTTGCATTCAGTGATGTAGT
GATACCAGCCTTGGCAATGCTAACAGTCTGTTGTTCCATAACTTCATGTATAGATGTA
CGATCA
Seq ID No. 52 (SNP base bold):
ATACCATGCAGGATTAGCTGCAGCAAGGACAGCAGTCCTTGCATTCAGTGATGTAGT
GATCCCAGCCTTGGCAATGCTAACAGTCTGTTGTTCCATAACTTCATGTATAGATGTA
CGATCA

QTL 27, as used herein, refers to a polymorphic genetic locus linked to genetic marker 07_32684348 in pea linkage group 2 (LG2) on chromosome 6. The two alleles of marker 07_32684348 at QTL 27 have the bases “A” or “T”, respectively, at position 259389351 of LG2, as set forth, respectively, in the nucleic acid sequences of Seq IDs 53 and 54. In cassette 14, QTL 27 may be homozygous for allele 1 (Seq ID 53) or be heterozygous (Seq IDs 53 and 54).

Seq ID No. 53 (SNP base bold):
GTCCCTAATGCTGCTTATGCTGGTGGTGGCCCAAGGAGTTCATGGCCCGCACAGGCT
CCCTCTGGCTATGGCTCTATGGGTTATGGAAACACTGCTCCTTGG
Seq ID No. 54 (SNP base bold):
GTCCCTAATGCTGCTTATGCTGGTGGTGGCCCAAGGAGTTCATGGCCCGCTCAGGCT
CCCTCTGGCTATGGCTCTATGGGTTATGGAAACACTGCTCCTTGG

QTL 28, as used herein, refers to a polymorphic genetic locus linked to genetic marker 42662_26712_871 in pea linkage group 1 (LG1) on chromosome 2. The two alleles of marker 42662_26712_871 at QTL 28 have the bases “T” or “C”, respectively, at position 410200645 of LG1, as set forth, respectively, in the nucleic acid sequences of Seq IDs 55 and 56. In cassettes 11 to 14, QTL 28 may be homozygous for allele 1 (Seq ID 55) or be heterozygous (Seq IDs 55 and 56).

Seq ID No. 55 (SNP base bold):
AGGTGGTGTTTCTGTTTTGTGTTCTTTACTTGGTCCTTTTACTTCATATGCTGTTGGTT
CTGAAGTTATTGGTATTCTTGTTAGTTTGACACTTGATTCTGAATCCAAAAAGAATCT
TATG
Seq ID No. 56 (SNP base bold):
AGGTGGTGTTTCTGTTTTGTGTTCTTTACTTGGTCCTTTTACTTCATATGCTGTTGGTT
CCGAAGTTATTGGTATTCTTGTTAGTTTGACACTTGATTCTGAATCCAAAAAGAATCT
TATG

QTL 29, as used herein, refers to a polymorphic genetic locus linked to genetic marker 044504_28363_461 in pea linkage group 6 (LG6) on chromosome 1. The two alleles of marker 044504_28363_461 at QTL 29 have the bases “T” or “C”, respectively, at position 167946502 of LG6, as set forth, respectively, in the nucleic acid sequences of Seq IDs 57 and 58. In cassettes 12 and 13, QTL 29 may be homozygous for allele 1 (Seq ID 57) or be heterozygous (Seq IDs 57 and 58).

Seq ID No. 57 (SNP base bold):
ACATATAATAGCACGTCGAAGATCTTCATCGTCCTTACTACAGAGCACTTGCACATA
TTGTATAAGGTTTGGAAACATCTCTTTTTCCGTTGTTGATGACAACGGAAAAAGAGA
CTTTTGT
Seq ID No. 58 (SNP base bold):
ACATATAATAGCACGTCGAAGATCTTCATCGTCCTTACTACAGAGCACTTGCACATA
TTGCATAAGGTTTGGAAACATCTCTTTTTCCGTTGTTGATGACAACGGAAAAAGAGA
CTTTTGT

QTL 30, as used herein, refers to a polymorphic genetic locus linked to genetic marker ER1 in pea linkage group 6 (LG6) on chromosome 1. The two alleles of marker ER1 at QTL 30 have the bases “C” or “G”, respectively, at position 175515672 of LG6, as set forth, respectively, in the nucleic acid sequences of Seq IDs 59 and 60. In cassette 11, QTL 30 may be homozygous for allele 2 (Seq ID 60) or be heterozygous (Seq IDs 59 and 18).

Seq ID No. 59 (SNP base bold):
GGTTTGCAAGGGACACAACATTTGGAAGAAGGCACTTGAGCATGTGGGCTCAGTCA
CCTATTTTGTTATGGATTGTAAGGGAACTTTTGTTACATAAAATTAATCATACACATT
AATTAAAT
Seq ID No. 60 (SNP base bold):
GGTTTGCAAGGGACACAACATTTGGAAGAAGGCACTTGAGCATGTGGGCTCAGTGA
CCTATTTTGTTATGGATTGTAAGGGAACTTTTGTTACATAAAATTAATCATACACATT
AATTAAAT

QTL 31, as used herein, refers to a polymorphic genetic locus linked to genetic marker 044835_28587_1878 in pea linkage group 1 (LG1) on chromosome 2. The two alleles of marker 044835_28587_1878 at QTL 31 have the bases “T” or “G”, respectively, at position 419557580 of LG1, as set forth, respectively, in the nucleic acid sequences of Seq IDs 61 and 62. In cassettes 11 to 14, QTL 31 may be homozygous for allele 1 (Seq ID 61) or be heterozygous (Seq IDs 61 and 62).

Seq ID No. 61 (SNP base bold):
TACATCAGTTTGAGAAAGTTACAGCAGAACTCACAACTCAAGAAGAAACTTGCAATT
TGTTATATCAACCGGAATTTCGCCAACGAGGTTTAAGTTGCTCAAATCCAGCAATTC
AAGCAGC
Seq ID No. 62 (SNP base bold):
TACATCAGTTTGAGAAAGTTACAGCAGAACTCACAACTCAAGAAGAAACTTGCAATT
TGTGATATCAACCGGAATTTCGCCAACGAGGTTTAAGTTGCTCAAATCCAGCAATTC
AAGCAGC

QTL 32, as used herein, refers to a polymorphic genetic locus linked to genetic marker 044855_28602_1561 in pea linkage group 1 (LG1) on chromosome 2. The two alleles of marker 044855_28602_1561 at QTL 32 have the bases “A” or “G”, respectively, at position 419560368 of LG1, as set forth, respectively, in the nucleic acid sequences of Seq IDs 63 and 64. In cassettes 11 to 14, QTL 32 may be homozygous for allele 1 (Seq ID 63) or be heterozygous (Seq IDs 63 and. 64).

Seq ID No. 63 (SNP base bold):
CATTACCTCACTTGACCAAGCCTTCAACCAAGCAAAGAAGCGTAGTCAAAAAGTTTG
TGGAGTTATAATATCAAACCCTTCAAACCCTACCGGAAAATTCTTAAATCGGGAAAC
ACTACTT
Seq ID No. 64 (SNP base bold):
CATTACCTCACTTGACCAAGCCTTCAACCAAGCAAAGAAGCGTAGTCAAAAAGTTTG
TGGGGTTATAATATCAAACCCTTCAAACCCTACCGGAAAATTCTTAAATCGGGAAAC
ACTACTT

Pea plants or part(s) thereof, as well as products such as commodity whole or split grains, pea protein concentrates (PPC), dairy analogues or protein-fortified beverages and meat analogues or texturized pea protein prepared therefrom, which have high protein content, low fat content and neutral taste are provided. The pea plans, part(s) and products are characterized by specific sets of nucleic acid genetic markers that correspond to identified quantitative trait loci (QTLs) related to specific phenotypic traits. In various embodiments, the phenotypic traits may comprise a high protein content of the seeds of at least 29% (weight %, dry matter base—DB), a low fat content of the seeds of at most 2% (weight %), and a neutral taste characterized by moderate to very mild off flavors in a qualitative tasting test and associated with reduced level of volatile compounds associated with off flavors. Phenotypic and genotypic analysis of many pea varieties was performed to derive the markers for these phenotypic traits, and a breeding simulation was used to identify the most common and most stable markers. Following verification of trait stability over several generations, markers and marker cassettes were defined as being uniquely present in the developed pea lines. The resulting pea lines can be used to provide products with pea-based protein and low or no off flavors, possibly without requiring additional chemical or biological processing. In various embodiments, the plants may be hybrids and/or the plant parts may comprise any of: a seed, an endosperm, an ovule, pollen, cell, cell culture, tissue culture, plant organ, protoplast, meristem, embryo, or a combination thereof.

In certain embodiments, the QTLs and markers associated therewith comprise at least three QTLs and corresponding markers, of at least one of the following cassettes: (i) cassette 1 comprising: the QTL and marker associated with the increased yield trait comprise QTL 1 with corresponding markers set forth in Seq. IDs 1 or 2, and the pea plant or part thereof is homozygous with respect to Seq. ID 1 or heterozygous at QTL 1, the QTL and marker associated with the low fat content trait comprise QTL 2 with corresponding markers set forth in Seq. IDs 3 or 4, and the pea plant or part thereof is homozygous with respect to Seq. ID 3 or heterozygous at QTL 2, and the QTL and marker associated with the increased yield trait comprise QTL 3 with corresponding markers set forth in Seq. IDs 5 or 6, and the pea plant or part thereof is homozygous with respect to Seq. ID 5 or heterozygous at QTL 3; (ii) at least one of cassettes 2, 3, 4, 8, 9 or 10 comprising the QTL and marker associated with the neutral taste trait comprise QTL 4 with corresponding markers set forth in Seq. IDs 7 or 8, and the pea plant or part thereof is homozygous with respect to Seq. ID 7 or heterozygous at QTL 4; and/or (iii) at least one of cassettes 5-10 comprising the QTL and marker associated with the neutral taste trait comprise QTL 13 with corresponding markers set forth in Seq. IDs 25 or 26, and the pea plant or part thereof is homozygous with respect to Seq. ID 25 or heterozygous at QTL 13.

For example, in certain embodiments, the pea plant(s) or part(s) thereof may comprise the QTLs and corresponding markers of cassette 1.

In certain embodiments, the pea plant(s) or part(s) thereof may comprise the QTLs and corresponding markers of at least one of cassettes 2, 3, 4, 8, 9 or 10, e.g., comprising QTL 4 as described herein.

For example, the QTLs and corresponding markers may comprise cassette 2 that further comprises: the QTL and marker associated with the neutral taste trait comprise QTL 6 with corresponding markers set forth in Seq. IDs 11 or 12, and the pea plant or part thereof is homozygous with respect to Seq. ID 11 or heterozygous at QTL 6, the QTL and marker associated with the neutral taste trait comprise QTL 11 with corresponding markers set forth in Seq. IDs 21 or 22, and the pea plant or part thereof is homozygous with respect to Seq. ID 21 or heterozygous at QTL 11, and the QTL and marker associated with the neutral taste trait comprise QTL 12 with corresponding markers set forth in Seq. IDs 23 or 23, and the pea plant or part thereof is homozygous with respect to Seq. ID 23 or heterozygous at QTL 12

For example, the QTLs and corresponding markers may comprise cassette 3 that further comprises the QTL and marker associated with the neutral taste trait comprise QTL 9 with corresponding markers set forth in Seq. IDs 17 or 18, and the pea plant or part thereof is homozygous with respect to Seq. ID 17 or heterozygous at QTL 9, and the QTL and marker associated with the neutral taste trait comprise QTL 10 with corresponding markers set forth in Seq. IDs 19 or 20, and the pea plant or part thereof is homozygous with respect to Seq. ID 19 or heterozygous at QTL 10.

For example, the QTLs and corresponding markers may comprise cassette 4 that further comprises the QTL and marker associated with the high protein trait comprise QTL 5 with corresponding markers set forth in Seq. IDs 9 or 10, and the pea plant or part thereof is homozygous with respect to Seq. ID 9 or heterozygous at QTL 5, the QTL and marker associated with the neutral taste trait comprise QTL 7 with corresponding markers set forth in Seq. IDs 13 or 14, and the pea plant or part thereof is homozygous with respect to Seq. ID 13 or heterozygous at QTL 7, and the QTL and marker associated with the neutral taste trait comprise QTL 8 with corresponding markers set forth in Seq. IDs 15 or 16, and the pea plant or part thereof is homozygous with respect to Seq. ID 15 or heterozygous at QTL 8.

For example, the QTLs and corresponding markers may comprise cassette 8 that further comprises the QTL and marker associated with the high yield trait comprise QTL 5 with corresponding markers set forth in Seq. IDs 9 or 10, and the pea plant or part thereof is homozygous with respect to Seq. ID 10 or heterozygous at QTL 5, the QTL and marker associated with the neutral taste trait comprise QTL 13 with corresponding markers set forth in Seq. IDs 25 or 26, and the pea plant or part thereof is homozygous with respect to Seq. ID 25 or heterozygous at QTL 13, and the QTL and marker associated with the neutral taste trait comprise QTL 21 with corresponding markers set forth in Seq. IDs 41 or 42, and the pea plant or part thereof is homozygous with respect to Seq. ID 41 or heterozygous at QTL 21.

For example, the QTLs and corresponding markers may comprise cassette 9 that further comprises the QTL and marker associated with the neutral taste trait comprise QTL 13 with corresponding markers set forth in Seq. IDs 25 or 26, and the pea plant or part thereof is homozygous with respect to Seq. ID 25 or heterozygous at QTL 13, the QTL and marker associated with the high protein trait comprise QTL 14 with corresponding markers set forth in Seq. IDs 27 or 28, and the pea plant or part thereof is homozygous with respect to Seq. ID 27 or heterozygous at QTL 14, and the QTL and marker associated with the high protein and the high yield traits comprise QTL 20 with corresponding markers set forth in Seq. IDs 39 or 40, and the pea plant or part thereof is homozygous with respect to Seq. ID 39 or heterozygous at QTL 20.

For example, the QTLs and corresponding markers may comprise cassette 10 that further comprises the QTL and marker associated with the neutral taste trait comprise QTL 6 with corresponding markers set forth in Seq. IDs 11 or 12, and the pea plant or part thereof is homozygous with respect to Seq. ID 11 or heterozygous at QTL 6, the QTL and marker associated with the neutral taste trait comprise QTL 13 with corresponding markers set forth in Seq. IDs 25 or 26, and the pea plant or part thereof is homozygous with respect to Seq. ID 25 or heterozygous at QTL 13, and the QTL and marker associated with the high protein and the high yield traits comprise QTL 20 with corresponding markers set forth in Seq. IDs 39 or 40, and the pea plant or part thereof is homozygous with respect to Seq. ID 39 or heterozygous at QTL 20.

In certain embodiments, the pea plant(s) or part(s) thereof may comprise the QTLs and corresponding markers of at least one of cassettes 5-10, e.g., comprising QTL 13 as described herein.

For example, the QTLs and corresponding markers may comprise cassette 5 that further comprises the QTL and marker associated with the high yield trait comprise QTL 5 with corresponding markers set forth in Seq. IDs 9 or 10, and the pea plant or part thereof is homozygous with respect to Seq. ID 10 or heterozygous at QTL 5, and the QTL and marker associated with the high protein trait comprise QTL 19 with corresponding markers set forth in Seq. IDs 37 or 38, and the pea plant or part thereof is homozygous with respect to Seq. ID 37 or heterozygous at QTL 19.

For example, the QTLs and corresponding markers may comprise cassette 6 that further comprises the QTL and marker associated with the neutral taste trait comprise QTL 16 with corresponding markers set forth in Seq. IDs 31 or 32, and the pea plant or part thereof is homozygous with respect to Seq. ID 31 or heterozygous at QTL 16, the QTL and marker associated with the high yield trait comprise QTL 17 with corresponding markers set forth in Seq. IDs 33 or 34, and the pea plant or part thereof is homozygous with respect to Seq. ID 33 or heterozygous at QTL 17, and the QTL and marker associated with the neutral taste trait comprise QTL 18 with corresponding markers set forth in Seq. IDs 35 or 36, and the pea plant or part thereof is homozygous with respect to Seq. ID 35 or heterozygous at QTL 18.

For example, the QTLs and corresponding markers may comprise cassette 7 that further comprises the QTL and marker associated with the high protein trait comprise QTL 14 with corresponding markers set forth in Seq. IDs 27 or 28, and the pea plant or part thereof is homozygous with respect to Seq. ID 27 or heterozygous at QTL 14, and the QTL and marker associated with the neutral taste trait comprise QTL 15 with corresponding markers set forth in Seq. IDs 29 or 30, and the pea plant or part thereof is homozygous with respect to Seq. ID 29 or heterozygous at QTL 15.

In various embodiments, commodity whole or split grains of the pea plants, pea protein concentrate (PPC) prepared from seeds of the pea plants, as well as dairy analogues, protein-fortified beverages, meat analogues or texturized pea protein comprising the PPC may comprise 29% w/w DB or more protein (dry base) and 2% w/w DB or less fat and have a neutral taste characterized by moderate to very mild off flavors in a qualitative tasting test and associated with reduced level of volatile compounds associated with off flavors.

Disclosed pea plant having high protein content, or part(s) thereof are provided. The pea plant comprises a plurality of loci associated with a corresponding plurality of QTLs having a corresponding plurality of nucleic acid genetic markers that are associated with a plurality of phenotypic traits of the pea plant, wherein the phenotypic traits comprise a high protein content of the seeds of at least 25% and semi-leafless and powdery mildew resistance, and wherein the plurality of QTLs and corresponding markers comprise at least two QTLs and corresponding markers.

In certain embodiments, the QTL and marker associated with the high protein trait comprise QTL 22 with corresponding marker set forth in Seq. IDs 43 or 44; the QTL and marker associated with the semi-leafless trait comprise QTL 28 with corresponding markers set forth in Seq. IDs 55 or 56; and the QTL and marker associated with the powdery mildew resistance trait comprise QTL 30 with corresponding markers set forth in Seq. IDs 59 or 60.

In certain embodiments, the QTL and marker associated with the high protein trait comprise QTL 22 with corresponding marker set forth in Seq. IDs 43 or 44; the QTL and marker associated with the semi-leafless trait comprise QTL 28 with corresponding markers set forth in Seq. IDs 55 or 56; and the QTL and marker associated with the powdery mildew resistance trait comprise QTL 29 with corresponding markers set forth in Seq. IDs 47 or 48.

In certain embodiments, the pea plant or part thereof may further comprise a QTL and marker associated with a protein composition trait that comprise QTL 23 with corresponding marker set forth in Seq. IDs 45 or 46.

In certain embodiments, the QTL and marker associated with the high protein trait comprise QTL 24 with corresponding marker set forth in Seq. IDs 47 or 48; and QTL 25 with corresponding marker set forth in Seq. IDs 49 or 50; the QTL and marker associated with the semi-leafless trait comprise QTL 28 with corresponding markers set forth in Seq. IDs 55 or 56; and the QTL and marker associated with the powdery mildew resistance trait comprise QTL 29 with corresponding markers set forth in Seq. IDs 57 or 58.

In certain embodiments, the QTL and marker associated with the high protein trait comprise QTL 26 with corresponding marker set forth in Seq. IDs 51 or 52; and the QTL and marker associated with the semi-leafless trait comprise QTL 28 with corresponding markers set forth in Seq. IDs 55 or 56. The pea plant or part thereof may further comprise a QTL and marker associated with a protein composition trait that comprise QTL 27 with corresponding marker set forth in Seq. IDs 53 or 54.

In certain embodiments, the phenotypic traits further comprise a protein composition trait and/or a yellow cotyledon trait. In certain embodiments, the plurality of QTLs and corresponding markers comprise at least three QTLs and corresponding markers. In certain embodiments, the phenotypic traits further comprise a yellow cotyledon trait, for example, the QTLs and markers associated with the yellow cotyledon trait comprise QTL 31 with corresponding markers set forth in Seq. IDs 61 or 62; and/or QTL 32 with corresponding markers set forth in Seq. IDs 63 or 64.

In certain embodiments, the phenotypic traits comprise a high protein content of the seeds of at least 25%, or possibly a high protein content of the seeds of at least 26%. In various embodiments, the plants may be hybrids and/or the plant parts may comprise any of: a seed, an endosperm, an ovule, pollen, cell, cell culture, tissue culture, plant organ, protoplast, meristem, embryo, or a combination thereof.

FIG. 4 is a high-level schematic illustration of a computationally supported breeding method 200, according to some embodiments of the invention. Method 200 may be at least partially implemented by at least one computer processor; computationally supported breeding method 200 is used to detect and combine QTLs from a plurality of pea varieties to develop the disclosed pea plants which are different than any of the parent varieties by virtue of the achieved phenotypical characteristics.

Computationally supported breeding method 200 comprises stages of trait discovery by growing and phenotyping a broad spectrum of varieties (stage 210), trait blending by developing hybridized lines through crossing the selected lines to mix and combine traits (stage 220) and selfing of the progeny in subsequent generations (stage 225), Target Product Genomic Code (TPGC) discovery by associating phenotypes and genotypes using derived linkage maps (stage 230), in silico selection (and validation) to suggest candidate varieties (stage 240), breeding candidate varieties (stage 250) and selecting varieties based on the best TPGC potential (stage 260), as explained in detail below. TPGC discovery (stage 230) and in silico selection and validation (stage 240) are based on computational algorithms that cannot be performed manually and provide the computational support for the judicious selection of the varieties that are generated and further crossed during the development process to yield disclosed pea plants. It is noted that during the discovery phase, the QTLs are derived in order to combine them (by hybridization) to create unique combinations of QTLs which do not exist in known world lines.

In certain embodiments, pea lines were bred to reach neutral taste (in addition to high protein content and low-fat content) by collecting various pea lines worldwide, creating F2 breeding populations, applying intensive phenotyping and genotyping of thousands of pea plants, predicting of QTLs that affect the taste and other phenotypical traits, and establishing unique QTL marker combinations, termed “marker cassettes” herein, to characterize novel lines provided by the methods described herein and not existing in commercial or natural lines.

The breeding methodology was based on algorithms for deriving the Target Product Genomic Code (TPGC) to associate (i) the Target Product (TP) being defined in advance based on market requirements and including a set of desired attributes (traits) that are available in natural genetic variations; and (ii) the Genomic Code (GC) comprising set(s) of genomic regions that include quantitative trait loci (QTLs) that affect and are linked to the TP traits. The algorithms may be configured to calculate multiple genomic interactions and to maximize the genomic potential of specific pea plants for the development of new pea varieties. The breeding program was constructed to derive the TPGC, and then by selfing, and optionally crossing, to achieve a pea product which contains the specific GC that corresponds to the required TPs.

Certain embodiments of the breeding process of developing lines, through crossing and successive generations of selfing comprise stages such as: (i) Trait Discovery, in which a broad spectrum of pea varieties from different geographies and worldwide sources are grown and phenotyped in order to identify new traits that can potentially be combined to create new pea varieties; (ii) Trait Blend, in which a crossing cycle is carried out based on phenotypic assumption(s), in which the different traits are mixed and combined. Initial trait cycle(s) are followed by additional cycle(s) to create F2 (and possibly higher generations) breeding population(s) that provide the basis for algorithmic analysis for constructing the TPGC; (iii) TPGC Discovery, in which the breeding populations of pea plant(s) are phenotyped and genotyped to produce linkage map(s), discover relevant QTLs and derive the TPGC; (iv) Trait TPGC Blend, in which accurate crossings are performed in order to calculate the most efficient way to reach the best TPGC. The crossings are performed after in silico selection and are based, at least in part, on phenotype assumptions, and (v) Consecutive algorithm-based GC discovery stage(s) applied to F2 (or higher generation) population(s) grown in additional cycle(s).

Defining the TP for high (or modified) protein and/or low fat pea varieties with neutral taste includes the development of high throughput methods for identifying high (or modified) protein and/or low fat level as well as neutral taste traits.

In the following non-limiting example of the breeding process, Trait Discovery (i) was based on proprietary germplasm including hundreds of elite varieties and 761 different pea lines that were obtained from the Germplasm Resources Information Network (GRIN) and the Israel Gene Bank (IGB). These lines were used for the Trait Blend stage (ii), with crosses executed based on the potential for enrichment of genomic diversity to create new complex(es) of traits for the high (or modified) protein and/or low fat level, as well as neutral taste—as the initial step for the TP-directed breeding program for high (or modified) protein and/or low fat neutrally-tasting pea lines. The resulting F1 hybrids were later self-crossed to create F2 breeding populations that showed phenotypic segregation. The F2 populations were then planted in two different environments for discovering the TPGC (iii) that includes high (or modified) protein, low fat and/or neutral taste traits. After screening and deep phenotyping of 3318 individuals from 11 populations, a set of ca. 400 representatives was selected in relation to neutral taste traits, and after screening 90,000 individuals, a set of ca. 3200 representatives was selected for high protein traits. The selected individuals from the F2 populations were further massively phenotyped for traits associated with high (or modified) protein, low fat and/or neutral taste, as detailed in the following description. The measurement results were summarized into the representative high (or modified) protein, low fat and/or neutral taste traits.

TPGC Discovery (iii) included genotyping ca. 2900 selected individual pea plants for neutral taste traits and ca. 3200 selected individual pea plants for high protein traits, from 8 populations. The analysis was performed with a panel of 600 markers based on single nucleotide polymorphisms (SNP) and directly designed based on the polymorphism found in the parental lines of the populations which were analyzed in depth using high throughput DNA sequencing technologies. The panel was designed to maximize the chance to have the largest number of common segregating SNPs in order to create highly similar linkage maps for all observed populations. The computation of linkage maps was executed on each linkage F2 population based on the genotyping results. Linkage maps were computed with ASMap package (Julian Taylor, David Butler (2017). R Package ASMap: Efficient Genetic Linkage Map Construction and Diagnosis. Journal of Statistical Software, 79(6), 1-29. doi:10.18637/jss.v079.i06), which runs in the R statistical environment (R Core Team (2023). R: A language and environment for statistical computing. R Foundation for Statistical Computing, Vienna, Austria. URL https://www.R-project.org/.). Discovery of QTLs that are related to high (or modified) protein, low fat and/or neutral taste traits was carried out with the R/qtl package (Broman et al. (2003) R/qtl: QTL mapping in experimental crosses. Bioinformatics 19:889-890 doi:10.1093/bioinformatics/btg112), based on the linkage maps and the F2 population phenotype data, and using the interval mapping algorithm (IM). The significance and co-occurrences of the markers for high protein level and protein content, low fat content and neutral taste traits were evaluated using an algorithm that related the genotype-phase of each marker to respective QTLs and phenotypic traits in linkage maps of the eight F2 populations (also called “linkage F2 populations” herein) in each population, for populations in different environments. QTL significance was computed with permutation, bootstrap tools and FDR (false discovery rate) for total analysis. The linkage maps of all eight F2 populations and the information of the high (or modified) protein, low fat and/or neutral taste traits over all genotyped plants belonging to those populations were analyzed and used to predict the QTLs in a “one trait to one marker” model, in which for all markers that constructed the linkage maps, each trait was tested independently against each one of the markers. In the provided examples, altogether 21 markers were found to be related to traits associated with high (or modified) protein, low fat and/or neutral taste components (see Table 1 above). The occurrence of some high (or modified) protein, low fat and/or neutral taste traits in two or more linkage maps of the F2 population (repetitive markers) strengthened its significance as representative for QTLs for these traits.

In the provided examples for high protein pea, 54 markers were found to be related to protein content and to protein composition, between 2-19 markers per population. Out of these markers, four markers (linked to corresponding QTLs 22, 24, 25, 26 in Table 1) were selected to use for identifying high protein lines and two markers (linked to QTL 23 and 27 in Table 1) were found to be associated with the protein composition trait (see also Table 2). In addition, one marker (linked to QTL 28 in Table 1) was found to be associated with the semi-leafless trait, two markers (linked to QTL 31 and 32 in Table 1) were found to be associated with the yellow cotyledon trait, and two markers (linked to QTLs 29 and 30 in Table 1) were found to be associated with the powdery mildew resistance trait.

In general, the eight linkage F2 populations presented different markers that related to high (or modified) protein, low fat and/or seed color. However, subsets of common markers were found to be shared by multiple populations and are referred to herein as marker cassettes.

It is emphasized that the breeding process is explained using non-limiting examples from a specific part of the breeding program, and is not limited to the specific populations and varieties derived by this specific part of the breeding program. For example, different F2 population may be bred and used to derive additional varieties that are characterized by one or more of the disclosed QTLs.

Following TPGC Discovery (iii), an in-silico breeding program (iv) was established to process the TPGC blend (including combinations of QTLs for different plants) to simulate and predict the genotypic states of self, cross-self and hybrid plant with respect to the QTLs and their predicted effects on each phase of the markers for the high (or modified) protein, low fat and/or neutral taste traits. The in-silico breeding program was constructed to yield millions of in silico selfing combinations which were bred and evaluated in-silico up to F4 to measure the potential for each of the genotyped plants to acquire the high (or modified) protein and/or low fat level in the right combination at the right phase. The analysis resulted in identifying ca. 393 F2 plants having the highest score for high (or modified) protein, low fat and/or seed color, which were then chosen for the actual selfing and cross-selfing procedures. The F3 seeds from these selected F2 plants were sown in plots in the subsequent growing season. Under this procedure, QTLs from different populations were combined to yield F3 plants containing new and unique cassettes of QTLs and resulting in high (or modified) protein, low fat and/or neutral taste.

The high (or modified) protein, low fat and/or neutral tasting pea lines were then validated as retaining the traits in the following generations by genotyping the F3 and some subsequent generation offspring to verify they maintained the identified marker cassettes. Specifically, the parental lines of linkage F2 populations together with 3108 different pea cultivars (landraces and old commercial varieties) were genotyped based on markers for the high (or modified) protein, low fat and/or neutral taste traits of all populations. The cassettes detailed in Table 1 were found to wholly differentiate the developed high (or modified) protein, low fat and/or neutral taste lines from the rest of the pea cultivars screened.

Disclosed pea lines that reach high protein content larger than 25%, e.g., in various lines, 26%, 27%, 28%, 30%, 35% or intermediate values (as dry weight percentage). Such high protein content allows using the disclosed pea lines for producing high protein concentrate (>65% dry weight percentage) for textured vegetable products (TVP) such as meat replacements. Moreover, advantageously, disclosed pea lines that enable the use of a sustainable and cost-effective protein enrichment process using dry fractionation and/or air classification as a processing method, which do not require large amounts of water and solvents (or even not requiring addition of any water or solvents, and having a significantly lower energy consumption) as the wet fractionation methods applied to prior art pea lines with lower protein content. Enabling dry fractionation to yield higher purity protein concentrate products also opens the possibility to use disclosed pea lines to produce highly nutritious and functional TVPs for human consumption, rather than the more common prior art use of pea concentrate for animal feed (typically 55% protein weight percent in commercial pea concentrates), due to their lower protein content and poor quality. Specifically, different disclosed pea lines were used to produce by dry fractionation texturized pea protein products having 63%, 64%, 66%, 68% and 72% protein. Generally, disclosed pea lines may be used to produce by dry fractionation texturized pea protein products having any of above 60%, above 65% or above 70% protein.

The inventors note that none of high (or modified) protein, low fat and/or neutral taste plants that were bred according to the methods described herein is naturally occurring; indeed, they were derived by highly complicated computationally-supported breeding methods 200 described above, in which the genotypes of multiple pea varieties were judiciously combined and analyzed, to discover and accumulate the recited QTL markers and corresponding phenotypic traits. Although the recited pea plants are not genetically modified by sequences originating from other species, they cannot be reached merely by natural processes, as is evident by the detailed and intentional breeding program that was applied to specifically measure required characteristics, detect corresponding markers using bioinformatics methods and combine the detected QTLs in the selected varieties by classic breeding approaches. The inventors note that due to the huge complexity of the breeding program, involving growing, selecting and breeding of hundreds of varieties over many generations in the field, and based on genetic analysis of the varieties and of the relations of QTL markers to phenotypic characteristics, this breeding process cannot happen merely by natural means and therefore cannot be considered a natural phenomenon. It is further noted that while the disclosed QTL markers are not heterologous to pea as a species, the identified QTLs are not present in the recited combinations in any of over 3108 landraces and old commercial varieties which were used as initial breeding stock, and that the QTL genomics of the pea plants has been significantly and judiciously modified by the breeding program. Therefore, at the taxonomic level of the pea varieties, the high (or modified) protein, low fat and/or neutrally tasting plants may be considered hybridized in that the QTL markers are mixed and introduced from other pea varieties.

In the above description, an embodiment is an example or implementation of the invention. The various appearances of “one embodiment”, “an embodiment”, “certain embodiments” or “some embodiments” do not necessarily all refer to the same embodiments. Although various features of the invention may be described in the context of a single embodiment, the features may also be provided separately or in any suitable combination. Conversely, although the invention may be described herein in the context of separate embodiments for clarity, the invention may also be implemented in a single embodiment. Certain embodiments of the invention may include features from different embodiments disclosed above, and certain embodiments may incorporate elements from other embodiments disclosed above. The disclosure of elements of the invention in the context of a specific embodiment is not to be taken as limiting their use in the specific embodiment alone. Furthermore, it is to be understood that the invention can be carried out or practiced in various ways and that the invention can be implemented in certain embodiments other than the ones outlined in the description above.

The invention is not limited to those diagrams or to the corresponding descriptions. For example, flow need not move through each illustrated box or state, or in exactly the same order as illustrated and described. Meanings of technical and scientific terms used herein are to be commonly understood as by one of ordinary skill in the art to which the invention belongs, unless otherwise defined. While the invention has been described with respect to a limited number of embodiments, these should not be construed as limitations on the scope of the invention, but rather as exemplifications of some of the preferred embodiments. Other possible variations, modifications, and applications are also within the scope of the invention. Accordingly, the scope of the invention should not be limited by what has thus far been described, but by the appended claims and their legal equivalents.

What is claimed is:

1. A pea plant or a part thereof that has high protein content, low fat content and neutral taste, the pea plant comprising:
a plurality of loci associated with a corresponding plurality quantitative trait loci (QTLs) having a corresponding plurality of nucleic acid genetic markers that are associated with a plurality of phenotypic traits of the pea plant, the phenotypic traits including high yield, high protein, low fat and neutral taste traits, wherein:
the phenotypic traits comprise a high protein content of the seeds of at least 29% w/w dry base, a low fat content of the seeds of at most 2% w/w dry base, and the neutral taste characterized by moderate to very mild off flavors in a qualitative tasting test and associated with reduced level of volatile compounds associated with off flavors,
the plurality of QTLs and corresponding markers comprise at least three QTLs and corresponding markers, of at least one of the following cassettes:
cassette 1 comprising:
the QTL and marker associated with the increased yield trait comprise QTL 1 with corresponding markers set forth in Seq. IDs 1 or 2, and the pea plant or part thereof is homozygous with respect to Seq. ID 1 or heterozygous at QTL 1,
the QTL and marker associated with the low fat content trait comprise QTL 2 with corresponding markers set forth in Seq. IDs 3 or 4, and the pea plant or part thereof is homozygous with respect to Seq. ID 3 or heterozygous at QTL 2, and
the QTL and marker associated with the increased yield trait and with a neutral taste trait comprise QTL 3 with corresponding markers set forth in Seq. IDs 5 or 6, and the pea plant or part thereof is homozygous with respect to Seq. ID 5 or heterozygous at QTL 3;
at least one of cassettes 2, 3, 4, 8, 9 or 10 comprising the QTL and marker associated with the neutral taste trait comprise QTL 4 with corresponding markers set forth in Seq. IDs 7 or 8, and the pea plant or part thereof is homozygous with respect to Seq. ID 7 or heterozygous at QTL 4; and/or
at least one of cassettes 5-10 comprising the QTL and marker associated with the neutral taste trait comprise QTL 13 with corresponding markers set forth in Seq. IDs 25 or 26, and the pea plant or part thereof is homozygous with respect to Seq. ID 25 or heterozygous at QTL 13.

2. The pea plant or a part thereof of claim 1, comprising the plurality of QTLs and corresponding markers of cassette 1.

3. The pea plant or a part thereof of claim 1, comprising the plurality of QTLs and corresponding markers of at least one of cassettes 2, 3, 4, 8, 9 or 10.

4. The pea plant or a part thereof of claim 3, comprising the plurality of QTLs and corresponding markers of cassette 2, further comprising:
the QTL and marker associated with the neutral taste trait comprise QTL 6 with corresponding markers set forth in Seq. IDs 11 or 12, and the pea plant or part thereof is homozygous with respect to Seq. ID 11 or heterozygous at QTL 6,
the QTL and marker associated with the neutral taste trait comprise QTL 11 with corresponding markers set forth in Seq. IDs 21 or 22, and the pea plant or part thereof is homozygous with respect to Seq. ID 21 or heterozygous at QTL 11, and
the QTL and marker associated with the neutral taste trait comprise QTL 12 with corresponding markers set forth in Seq. IDs 23 or 23, and the pea plant or part thereof is homozygous with respect to Seq. ID 23 or heterozygous at QTL 12.

5. The pea plant or a part thereof of claim 3, comprising the plurality of QTLs and corresponding markers of cassette 3, further comprising:
the QTL and marker associated with the neutral taste trait comprise QTL 9 with corresponding markers set forth in Seq. IDs 17 or 18, and the pea plant or part thereof is homozygous with respect to Seq. ID 17 or heterozygous at QTL 9, and
the QTL and marker associated with the neutral taste trait comprise QTL 10 with corresponding markers set forth in Seq. IDs 19 or 20, and the pea plant or part thereof is homozygous with respect to Seq. ID 19 or heterozygous at QTL 10.

6. The pea plant or a part thereof of claim 3, comprising the plurality of QTLs and corresponding markers of cassette 4, further comprising:
the QTL and marker associated with the high protein trait comprise QTL 5 with corresponding markers set forth in Seq. IDs 9 or 10, and the pea plant or part thereof is homozygous with respect to Seq. ID 9 or heterozygous at QTL 5,
the QTL and marker associated with the neutral taste trait comprise QTL 7 with corresponding markers set forth in Seq. IDs 13 or 14, and the pea plant or part thereof is homozygous with respect to Seq. ID 13 or heterozygous at QTL 7, and
the QTL and marker associated with the neutral taste trait comprise QTL 8 with corresponding markers set forth in Seq. IDs 15 or 16, and the pea plant or part thereof is homozygous with respect to Seq. ID 15 or heterozygous at QTL 8.

7. The pea plant or a part thereof of claim 3, comprising the plurality of QTLs and corresponding markers of cassette 8, further comprising:
the QTL and marker associated with the high yield trait comprise QTL 5 with corresponding markers set forth in Seq. IDs 9 or 10, and the pea plant or part thereof is homozygous with respect to Seq. ID 10 or heterozygous at QTL 5,
the QTL and marker associated with the neutral taste trait comprise QTL 13 with corresponding markers set forth in Seq. IDs 25 or 26, and the pea plant or part thereof is homozygous with respect to Seq. ID 25 or heterozygous at QTL 13, and
the QTL and marker associated with the neutral taste trait comprise QTL 21 with corresponding markers set forth in Seq. IDs 41 or 42, and the pea plant or part thereof is homozygous with respect to Seq. ID 41 or heterozygous at QTL 21.

8. The pea plant or a part thereof of claim 3, comprising the plurality of QTLs and corresponding markers of cassette 9, further comprising:
the QTL and marker associated with the neutral taste trait comprise QTL 13 with corresponding markers set forth in Seq. IDs 25 or 26, and the pea plant or part thereof is homozygous with respect to Seq. ID 25 or heterozygous at QTL 13,
the QTL and marker associated with the high protein trait comprise QTL 14 with corresponding markers set forth in Seq. IDs 27 or 28, and the pea plant or part thereof is homozygous with respect to Seq. ID 27 or heterozygous at QTL 14, and
the QTL and marker associated with the high protein and the high yield traits comprise QTL 20 with corresponding markers set forth in Seq. IDs 39 or 40, and the pea plant or part thereof is homozygous with respect to Seq. ID 39 or heterozygous at QTL 20.

9. The pea plant or a part thereof of claim 3, comprising the plurality of QTLs and corresponding markers of cassette 10, further comprising:
the QTL and marker associated with the neutral taste trait comprise QTL 6 with corresponding markers set forth in Seq. IDs 11 or 12, and the pea plant or part thereof is homozygous with respect to Seq. ID 11 or heterozygous at QTL 6,
the QTL and marker associated with the neutral taste trait comprise QTL 13 with corresponding markers set forth in Seq. IDs 25 or 26, and the pea plant or part thereof is homozygous with respect to Seq. ID 25 or heterozygous at QTL 13, and
the QTL and marker associated with the high protein and the high yield traits comprise QTL 20 with corresponding markers set forth in Seq. IDs 39 or 40, and the pea plant or part thereof is homozygous with respect to Seq. ID 39 or heterozygous at QTL 20.

10. The pea plant or a part thereof of claim 1, comprising the plurality of QTLs and corresponding markers of at least one of cassettes 5-10.

11. The pea plant or a part thereof of claim 10, comprising the plurality of QTLs and corresponding markers of cassette 5, further comprising:
the QTL and marker associated with the high yield trait comprise QTL 5 with corresponding markers set forth in Seq. IDs 9 or 10, and the pea plant or part thereof is homozygous with respect to Seq. ID 10 or heterozygous at QTL 5, and
the QTL and marker associated with the high protein trait comprise QTL 19 with corresponding markers set forth in Seq. IDs 37 or 38, and the pea plant or part thereof is homozygous with respect to Seq. ID 37 or heterozygous at QTL 19.

12. The pea plant or a part thereof of claim 10, comprising the plurality of QTLs and corresponding markers of cassette 6, further comprising:
the QTL and marker associated with the neutral taste trait comprise QTL 16 with corresponding markers set forth in Seq. IDs 31 or 32, and the pea plant or part thereof is homozygous with respect to Seq. ID 31 or heterozygous at QTL 16,
the QTL and marker associated with the high yield trait comprise QTL 17 with corresponding markers set forth in Seq. IDs 33 or 34, and the pea plant or part thereof is homozygous with respect to Seq. ID 33 or heterozygous at QTL 17, and
the QTL and marker associated with the neutral taste trait comprise QTL 18 with corresponding markers set forth in Seq. IDs 35 or 36, and the pea plant or part thereof is homozygous with respect to Seq. ID 35 or heterozygous at QTL 18.

13. The pea plant or a part thereof of claim 10, comprising the plurality of QTLs and corresponding markers of cassette 7, further comprising:
the QTL and marker associated with the high protein trait comprise QTL 14 with corresponding markers set forth in Seq. IDs 27 or 28, and the pea plant or part thereof is homozygous with respect to Seq. ID 27 or heterozygous at QTL 14, and
the QTL and marker associated with the neutral taste trait comprise QTL 15 with corresponding markers set forth in Seq. IDs 29 or 30, and the pea plant or part thereof is homozygous with respect to Seq. ID 29 or heterozygous at QTL 15.

14. Commodity whole or split grains of the pea plants of claim 1, comprising 29% w/w or more protein (dry base) and 2% w/w or less fat (dry base) and neutral taste characterized by moderate to very mild off flavors in a qualitative tasting test and associated with reduced level of volatile compounds associated with off flavors.

15. Pea protein concentrate (PPC) prepared from seeds of the pea plants of claim 1, comprising 29% w/w or more protein (dry base) and 2% w/w or less fat (dry base) and neutral taste characterized by moderate to very mild off flavors in a qualitative tasting test and associated with reduced level of volatile compounds associated with off flavors.

16. A dairy analogue or protein-fortified beverage comprising the PPC of claim 15 and being taste neutral characterized by moderate to very mild off flavors in a qualitative tasting test and associated with reduced level of volatile compounds associated with off flavors.

17. A meat analogue or texturized pea protein comprising the PPC of claim 15 and being taste neutral characterized by moderate to very mild off flavors in a qualitative tasting test and associated with reduced level of volatile compounds associated with off flavors.

18. A pea plant or a part thereof that has high protein content, the pea plant comprising:
a plurality of loci associated with a corresponding plurality quantitative trait loci (QTLs) having a corresponding plurality of nucleic acid genetic markers that are associated with a plurality of phenotypic traits of the pea plant, wherein:
the phenotypic traits comprise a high protein content of the seeds of at least 25% and semi-leafless and powdery mildew resistance,
the plurality of QTLs and corresponding markers comprise at least three QTLs and corresponding markers,
the QTL and marker associated with the semi-leafless trait comprise QTL 28 with corresponding markers set forth in Seq. IDs 55 or 56, and
the pea plant or part thereof is homozygous with respect to Seq. ID 55 or heterozygous at QTL 28.

19. The pea plant or a part thereof according to claim 18, wherein:
the QTL and marker associated with the high protein trait comprise QTL 22 with corresponding marker set forth in Seq. IDs 43 or 44,
the pea plant or part thereof comprise QTL 23 with corresponding marker set forth in Seq. IDs 45 or 46,
the QTL and marker associated with the powdery mildew resistance trait comprise QTL 30 with corresponding markers set forth in Seq. IDs 59 or 60,
the pea plant or part thereof is homozygous with respect to Seq. ID 44 or heterozygous at QTL 22,
the pea plant or part thereof is homozygous with respect to Seq. ID 45 or heterozygous at QTL 23, and
the pea plant or part thereof is homozygous with respect to Seq. ID 60 or heterozygous at QTL 30.

20. The pea plant or a part thereof according to claim 18, wherein:
the QTL and marker associated with the high protein trait comprise QTL 22 with corresponding marker set forth in Seq. IDs 43 or 44,
the pea plant or part thereof comprise QTL 23 with corresponding marker set forth in Seq. IDs 45 or 46,
the QTL and marker associated with the powdery mildew resistance trait comprise QTL 29 with corresponding markers set forth in Seq. IDs 57 or 58,
the pea plant or part thereof is homozygous with respect to Seq. ID 22 or heterozygous at QTL 22,
the pea plant or part thereof is homozygous with respect to Seq. ID 25 or heterozygous at QTL 23, and
the pea plant or part thereof is homozygous with respect to Seq. ID 36 or heterozygous at QTL 29.
